(19) United States
(12) Patent Application Publication
(10) Pub. No.: US 2024/0343753 A1
(43) Pub. Date: Oct. 17, 2024

(54) THERAPEUTIC COMPOUNDS

(71) Applicant: Regents of the University of Minnesota, Minneapolis, MN (US)

(72) Inventor: Natalia Tretyakova, Minneapolis, MN (US)

(73) Assignee: Regents of the University of Minnesota, Minneapolis, MN (US)

(21) Appl. No.: 18/633,908

(22) Filed: Apr. 12, 2024

Related U.S. Application Data
(60) Provisional application No. 63/458,815, filed on Apr. 12, 2023.

(51) Int. Cl. C07H 19/073; A61K 31/7068

(52) U.S. Cl. CPC C07H 19/073; A61K 31/7068

(57) ABSTRACT

The invention provides a compound of formula (I):

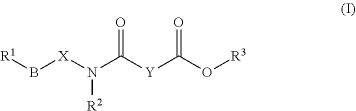

or a salt thereof, wherein R¹, R², R³, B, X, and Y have any of the values described in the specification, as well as compositions comprising a compound of formula (I). The compounds are useful as TET inhibitors, epigenetic modulators, and for treating cancer, eating disorders, and as adjuvants in cancer immunotherapy.

### CROSS REFERENCE TO RELATED APPLICATION

This application claims priority to U.S. Provisional Application No. 63/458,815, filed on 12 Apr. 2023. The entire content of this United States Provisional Application is hereby incorporated by reference herein.

### STATEMENT REGARDING FEDERALLY SPONSORED RESEARCH

This invention was made with government support under CA095039 awarded by the National Institutes of Health. The government has certain rights in the invention.

### BACKGROUND

DNA methylation is a chemical change to the bases of DNA. This modification plays important roles in various biological processes as it regulates gene expression, genome stability, genomic imprinting, and cellular differentiation. The most abundant DNA methylation mark is 5-methylcytosine (5mC). 5mC modifications are introduced enzymatically by DNA methyltransferases (DNMTs) and are actively removed via a pathway initiated by ten-eleven translocation (TET) dioxygenases.

TET proteins catalyze an α-ketoglutarate (α-KG)-mediated oxidation of 5mC to 5-hydroxymethyl-cytosine (5hmC), 5-formylcytosine (5fC) and 5-carboxylcytosine (5caC). TET enzymes regulate gene transcription, guide embryonic development, and prevent oncogenesis by controlling the levels of mC through DNA demethylation. In many cancers, TET expression and activity are dysregulated. In chronic lymphocytic leukemia, elevated levels of 5hmC and increased expression of TET2 and TET3 are observed. Additionally, TET1 is overexpressed in about 40% of patients with triple negative breast cancer (Cancer Res (2018) 78 (15): 4126-4137). TET2 is frequently mutated in patients with acute myeloid leukemia (Clin Exp Med. (2024) 24 (1): 35), and these mutations have been used for prognostic purposes. Additionally, TET disruption could be a useful approach in CAR-T cell immunotherapy (Nature. 2018 June; 558 (7709): 307-312).

Given TETs central role in the above-mentioned cancers and cancer therapy, TET proteins have the potential to be therapeutic targets. However, this area is underexplored. Potent and selective small molecule TET protein inhibitors are lacking; current reported TET inhibitors possess liabilities that limit their therapeutic utility, such as, for example, sub-optimal potency and/or potential off-target effects (Guan, Yihong, et al., The Journal of Clinical Investigation (2022); Chua, Gabriella N L, et al. ACS medicinal chemistry letters 10.2 (2019): 180-185; Guan, Yihong, et al., Blood cancer discovery 2.2 (2021): 146; Tiwari, Anand D., et al. Bioorganic & Medicinal Chemistry 39 (2021): 116141; Singh, Anup Kumar, et al., Proceedings of the National Academy of Sciences 117.7 (2020): 3621-3626; Weirath, Nicholas A., et al., ACS Medicinal Chemistry Letters (2022); WO2019108796A1; and US2020316069A1).

TET dioxygenase inhibitors may also be useful got treating eating disorders, such as, for example, annorexia (Proc Natl Acad Sci USA (2023) 120 (16): e2300015120).

Currently there is a need for TET dioxygenase inhibitors. Potent and selective TET inhibitors could be used as tool compounds in epigenetic research to elucidate the roles TET proteins play in human disease. TET inhibitors could also have potential utility as therapeutic agents for treating blood and breast cancers, eating disorders, and as adjuvants in cancer immunotherapy.

### SUMMARY

Compounds that inhibit TET enzyme activity are provided. Representative compounds have low-mid μM inhibitory concentrations. Representative compounds have also been shown to block the enzymatic oxidation of TET in vitro and modulate TET activity in a human cell based assay

Accordingly, in one aspect the present invention provides A compound of formula (I):

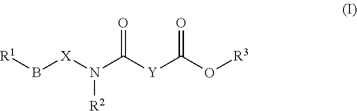

or a salt thereof, wherein:
R¹ is:

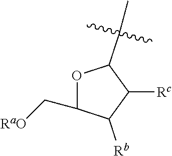

B is selected from the group consisting of:

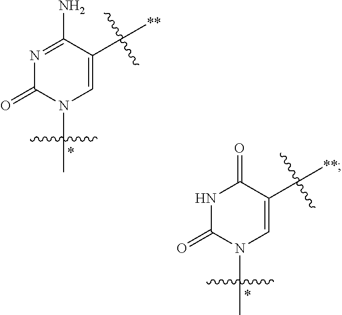

wherein * designates the point of attachment to R¹ and ** designates the point of attachment with X;
X is (C₁-C₆)alkyl or (C₂-C₆)alkynyl;
R² is H, OH, or (C₁-C₃)alkoxy;
Y is (C₁-C₆)alkyl or (C₂-C₆)alkenyl;
R³ is H or (C₁-C₆)alkyl;
R^{a} is H;
R^{b} is OH; and
R^{c} is H.

The invention also provides a pharmaceutical composition comprising a compound of formula (I) or a pharmaceutically acceptable salt thereof and a pharmaceutically acceptable excipient.

The invention also provides a method to treat cancer in an animal, comprising administering a compound of formula (I) or a pharmaceutically acceptable salt thereof to the animal.

The invention also provides a method to treat a neurological disorder (e.g., Alzheimer's disease) in an animal, comprising administering a compound of formula (I) or a pharmaceutically acceptable salt thereof to the animal.

The invention also provides a method to treat an eating disorder (e.g., anorexia) in an animal, comprising administering a compound of formula (I) or a pharmaceutically acceptable salt thereof to the animal.

The invention also provides a method to improve the outcome of an immunotherapy, comprising administering the immunotherapy to an animal in combination with a compound of formula (I) or a pharmaceutically acceptable salt thereof.

The invention also provides a method to modulate DNA demethylation, comprising inhibition of a TET enzyme with a compound of formula (I) or a salt thereof in vitro or in vivo.

The invention also provides a method to inhibit TET activity in vitro or in vivo comprising contacting a TET enzyme with a compound of formula (I) or a salt thereof.

The invention also provides a method to improve the efficacy of immunotherapy for cancer in an animal, comprising administering a compound of formula (I) or a pharmaceutically acceptable salt thereof to the animal in combination with immunotherapy.

The invention also provides processes and intermediates disclosed herein that are useful for preparing a compound of formula (I) or a salt thereof.

### BRIEF DESCRIPTION OF DRAWINGS

FIG. 1A and FIG. 1B: Shows preliminary screen of TET inhibitors in leukemia cells to assess effects of global hmC levels. Representative dot blot for 5hmC following treatment of K562 with 5 μM of specified inhibitor for 24 hours and extraction of cells genomic DNA; top: anti 5hmC antibody signal; bottom: methylene blue staining for B) Quantified hmC blotting intensity via densitometry normalized to DMSO treatment. Data represent mean±SD of two biological replicates. (Example 2)

FIG. 2: Shows the effects of TET inhibitor treatment on recombinant TET1 activity. 5mC containing DNA was incubated with the TET1 catalytic domain for 30 min in the presence or in the absence of the specified inhibitor in DMSO. The formation of hmC was detected by a quantitative LC-MS assay and is displayed as % activity relative to DMSO controls. All data are averaged triplicates normalized to DMSO±SD. (Example 2).

FIG. 3: Shows the effects of TET inhibitor treatment on recombinant TET2 activity. 5mC containing DNA was incubated with the TET1 catalytic domain for 30 min in the presence or in the absence of inhibitor 13b in DMSO. The loss of mC was detected by a quantitative LC-MS assay and is displayed as % activity relative to DMSO controls. All data are averaged triplicates normalized to DMSO±SD. (Example 2).

FIG. 4A and FIG. 4B: Show data from Example 3.

### DETAILED DESCRIPTION OF THE INVENTION

The following definitions are used, unless otherwise described: halo or halogen is fluoro, chloro, bromo, or iodo. Alkyl, alkoxy, alkenyl, alkynyl, etc. denote both straight and branched groups; but reference to an individual radical such as propyl embraces only the straight chain radical, a branched chain isomer such as isopropyl being specifically referred to.

The term “alkyl”, by itself or as part of another substituent, means, unless otherwise stated, a straight or branched chain hydrocarbon radical, having the number of carbon atoms designated (i.e., C₁₋₈ means one to eight carbons). Examples include (C₁-C₈)alkyl, (C₂-C₈)alkyl, C₁-C₆)alkyl, (C₂-C₆)alkyl and (C₃-C₆)alkyl. Examples of alkyl groups include methyl, ethyl, n-propyl, iso-propyl, n-butyl, t-butyl, iso-butyl, sec-butyl, n-pentyl, n-hexyl, n-heptyl, n-octyl, and higher homologs and isomers.

The term “alkenyl” refers to an unsaturated alkyl radical having one or more double bonds. Examples of such unsaturated alkyl groups include vinyl, 2-propenyl, crotyl, 2-isopentenyl, 2-(butadienyl), 2,4-pentadienyl, 3-(1,4-pentadienyl) and the higher homologs and isomers.

The term “alkynyl” refers to an unsaturated alkyl radical having one or more triple bonds. Examples of such unsaturated alkyl groups ethynyl, 1- and 3-propynyl, 3-butynyl, and higher homologs and isomers.

The term “alkoxy” refers to an alkyl groups attached to the remainder of the molecule via an oxygen atom (“oxy”).

As used herein, the term “protecting group” refers to a substituent that is commonly employed to block or protect a particular functional group on a compound. For example, an “amino-protecting group” is a substituent attached to an amino group that blocks or protects the amino functionality in the compound. Suitable amino-protecting groups include acetyl, trifluoroacetyl, t-butoxycarbonyl (BOC), benzyloxycarbonyl (CBZ) and 9-fluorenylmethylenoxycarbonyl (Fmoc). Similarly, a “hydroxy-protecting group” refers to a substituent of a hydroxy group that blocks or protects the hydroxy functionality. Suitable protecting groups include acetyl and silyl. A “carboxy-protecting group” refers to a substituent of the carboxy group that blocks or protects the carboxy functionality. Common carboxy-protecting groups include phenylsulfonylethyl, cyanoethyl, 2-(trimethylsilyl)ethyl, 2-(trimethylsilyl) ethoxymethyl, 2-(p-toluenesulfonyl)ethyl, 2-(p-nitrophenylsulfenyl)ethyl, 2-(diphenylphosphino)-ethyl, nitroethyl and the like. For a general description of protecting groups and their use, see P.G.M. Wuts and T.W. Greene, Greene's Protective Groups in Organic Synthesis 4^{th} edition, Wiley-Interscience, New York, 2006.

As used herein a wavy line “ [symbol] ” that intersects a bond in a chemical structure indicates the point of attachment of the bond that the wavy bond intersects in the chemical structure to the remainder of a molecule.

The terms “treat”, “treatment”, or “treating” to the extent it relates to a disease or condition includes inhibiting the disease or condition, eliminating the disease or condition, and/or relieving one or more symptoms of the disease or condition. The terms “treat”, “treatment”, or “treating” also refer to both therapeutic treatment and/or prophylactic treatment or preventative measures, wherein the object is to prevent or slow down (lessen) an undesired physiological change or disorder, such as, for example, the development or spread of cancer. For example, beneficial or desired clinical results include, but are not limited to, alleviation of symptoms, diminishment of extent of disease or disorder, stabilized (i.e., not worsening) state of disease or disorder, delay or slowing of disease progression, amelioration or palliation of the disease state or disorder, and remission (whether partial or total), whether detectable or undetectable. “Treat”, “treatment”, or “treating,” can also mean prolonging survival as compared to expected survival if not receiving treatment. Those in need of treatment include those already with the disease or disorder as well as those prone to have the disease or disorder or those in which the disease or disorder is to be prevented. In one embodiment “treat”, “treatment”, or “treating” does not include preventing or prevention,

The phrase “therapeutically effective amount” or “effective amount” includes but is not limited to an amount of a compound of the that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

The term “animal” as used herein includes mammals, fish, birds, reptiles, and amphibians.

The term “mammal” as used herein refers to humans, higher non-human primates, rodents, domestic, cows, horses, pigs, sheep, dogs and cats. In one embodiment, the mammal is a human. The term “patient” as used herein refers to any animal including mammals. In one embodiment, the patient is a mammalian patient. In one embodiment, the patient is a human patient.

The compounds disclosed herein can also exist as tautomeric isomers in certain cases. Although only one delocalized resonance structure may be depicted, all such forms are contemplated within the scope of the invention.

It is understood by one skilled in the art that this invention also includes any compound claimed that may be enriched at any or all atoms above naturally occurring isotopic ratios with one or more isotopes such as, but not limited to, deuterium (²H or D). As a non-limiting example, a —CH₃ group may be substituted with —CD₃.

The pharmaceutical compositions of the invention can comprise one or more excipients. When used in combination with the pharmaceutical compositions of the invention the term “excipients” refers generally to an additional ingredient that is combined with the compound of formula (I) or the pharmaceutically acceptable salt thereof to provide a corresponding composition. For example, when used in combination with the pharmaceutical compositions of the invention the term “excipients” includes, but is not limited to: carriers, binders, disintegrating agents, lubricants, sweetening agents, flavoring agents, coatings, preservatives, and dyes.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the invention can contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof such as racemic mixtures, form part of the present invention. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer can also be referred to as an enantiomer, and a mixture of such isomers is often called an enantiomeric mixture. A 50:50 mixture of enantiomers is referred to as a racemic mixture or a racemate, which can occur where there has been no stereoselection or stereospecificity in a chemical reaction or process. The terms “racemic mixture” and “racemate” refer to an equimolar mixture of two enantiomeric species, devoid of optical activity.

It will be appreciated by those skilled in the art that compounds of the invention having a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase).

When a bond in a compound formula herein is drawn in a non-stereochemical manner (e.g. flat), the atom to which the bond is attached includes all stereochemical possibilities. When a bond in a compound formula herein is drawn in a defined stereochemical manner (e.g. bold, bold-wedge, dashed or dashed-wedge), it is to be understood that the atom to which the stereochemical bond is attached is enriched in the absolute stereoisomer depicted unless otherwise noted. In one embodiment, the compound may be at least 51% the absolute stereoisomer depicted. In another embodiment, the compound may be at least 60% the absolute stereoisomer depicted. In another embodiment, the compound may be at least 80% the absolute stereoisomer depicted. In another embodiment, the compound may be at least 90% the absolute stereoisomer depicted. In another embodiment, the compound may be at least 95 the absolute stereoisomer depicted. In another embodiment, the compound may be at least 99% the absolute stereoisomer depicted.

Specific values listed below for radicals, substituents, and ranges, are for illustration only; they do not exclude other defined values or other values within defined ranges for the radicals and substituents. It is to be understood that two or more values may be combined. It is also to be understood that the values listed herein below (or subsets thereof) can be excluded.

Specifically, (C₁-C₆)alkyl can be methyl, ethyl, propyl, isopropyl, butyl, iso-butyl, sec-butyl, pentyl, 3-pentyl, or hexyl; (C₁-C₆)alkoxy can be methoxy, ethoxy, propoxy, isopropoxy, butoxy, iso-butoxy, sec-butoxy, pentoxy, 3-pentoxy, or hexyloxy; (C₂-C₆)alkenyl can be vinyl, allyl, 1-propenyl, 2-propenyl, 1-butenyl, 2-butenyl, 3-butenyl, 1,-pentenyl, 2-pentenyl, 3-pentenyl, 4-pentenyl, 1-hexenyl, 2-hexenyl, 3-hexenyl, 4-hexenyl, or 5-hexenyl; and C₂-C₆)alkynyl can be ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, or 5-hexynyl.

A specific compound or salt is a compound of formula (Ia):

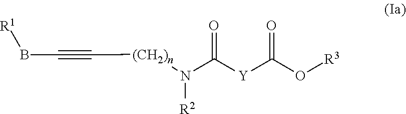

or a salt thereof, wherein n is 1, 2, 3, or 4.
A specific value for X is (C₁-C₆)alkyl.
A specific value for X is (C₂-C₆)alkynyl.
A specific value for R² is H.
A specific value for R² is OH.
A specific value for R² is (C₁-C₃)alkoxy.
A specific value for Y is (C₁-C₆)alkyl.
A specific value for Y is (C₂-C₆)alkenyl.
A specific value for R³ is H.
A specific value for R³ is (C₁-C₆)alkyl.

A specific compound or salt is selected from the group consisting of:
methyl (E)-4-((4-(4-amino-1-((2R,4S,5R)-4-hydroxy-5-(hydroxymethyl)-tetrahydrofuran-2-yl)-2-oxo-1,2-dihydropyrimidin-5-yl)but-3-yn-1-yl)amino)-4-oxobut-2-enoate (7b);
methyl (E)-4-((4-(1-((2R,4S,5R)-4-hydroxy-5-(hydroxymethyl)tetrahydrofuran-2-yl)-2,4-dioxo-1,2,3,4-tetrahydropyrimidin-5-yl)but-3-yn-1-yl)amino)-4-oxobut-2-enoate (9b);
methyl 4-((3-(4-amino-1-((2R,4S,5R)-4-hydroxy-5-(hydroxymethyl)-tetrahydrofuran-2-yl)-2-oxo-1,2-dihydropyrimidin-5-yl) prop-2-yn-1-yl)(hydroxy)amino)-4-oxobutanoate (10a);
methyl 4-((4-(4-amino-1-((2R,4S,5R)-4-hydroxy-5-(hydroxymethyl)-tetrahydrofuran-2-yl)-2-oxo-1,2-dihydropyrimidin-5-yl)but-3-yn-1-yl)(hydroxy)amino)-4-oxobutanoate (10b);
methyl (E)-4-((4-(4-amino-1-((2R,4S,5R)-4-hydroxy-5-(hydroxymethyl)-tetrahydrofuran-2-yl)-2-oxo-1,2-dihydropyrimidin-5-yl)but-3-yn-1-yl)(hydroxy)amino)-4-oxobut-2-enoate (11); and
methyl (E)-4-(hydroxy (4-(1-((2R,4S,5R)-4-hydroxy-5-(hydroxymethyl)-tetrahydrofuran-2-yl)-2,4-dioxo-1,2,3,4-tetrahydropyrimidin-5-yl)but-3-yn-1-yl)amino)-4-oxobut-2-enoate (12);
and salts thereof.

Another specific compound or salt is selected from the group consisting of:
4-((3-(4-amino-1-((2R,4S,5R)-4-hydroxy-5-(hydroxymethyl)tetrahydrofuran-2-yl)-2-oxo-1,2-dihydropyrimidin-5-yl) prop-2-yn-1-yl)(hydroxy)amino)-4-oxobutanoic acid (13a);
4-((4-(4-amino-1-((2R,4S,5R)-4-hydroxy-5-(hydroxymethyl)tetrahydrofuran-2-yl)-2-oxo-1,2-dihydropyrimidin-5-yl)but-3-yn-1-yl)(hydroxy)amino)-4-oxobutanoic acid (13b);
(E)-4-((4-(4-amino-1-((2R,4S,5R)-4-hydroxy-5-(hydroxymethyl)tetrahydrofuran-2-yl)-2-oxo-1,2-dihydropyrimidin-5-yl)but-3-yn-1-yl)amino)-4-oxobut-2-enoic acid (14);
(E)-4-((4-(4-amino-1-((2R,4S,5R)-4-hydroxy-5-(hydroxymethyl)tetrahydrofuran-2-yl)-2-oxo-1,2-dihydropyrimidin-5-yl)but-3-yn-1-yl)(hydroxy)amino)-4-oxobut-2-enoic acid (15);
(E)-4-((4-(1-((2R,4S,5R)-4-hydroxy-5-(hydroxymethyl)tetrahydrofuran-2-yl)-2,4-dioxo-1,2,3,4-tetrahydropyrimidin-5-yl)but-3-yn-1-yl)amino)-4-oxobut-2-enoic acid (16);
(E)-4-(hydroxy (4-(1-((2R,4S,5R)-4-hydroxy-5-(hydroxymethyl)tetrahydrofuran-2-yl)-2,4-dioxo-1,2,3,4-tetrahydropyrimidin-5-yl)but-3-yn-1-yl)amino)-4-oxobut-2-enoic acid (17); and
4-((3-(4-amino-1-((2R,4S,5R)-4-hydroxy-5-(hydroxymethyl)tetrahydrofuran-2-yl)-2-oxo-1,2-dihydropyrimidin-5-yl) propyl)(hydroxy)amino)-4-oxobutanoic acid (18);
and salts thereof.

In one embodiment, the compound of formula (I) is not:

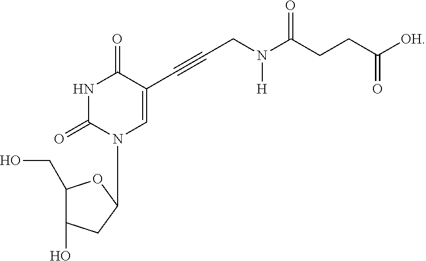 [SMILES: O=C(O)CCC(=O)NCC#Cc1cn(C2CC(O)C(CO)O2)c(=O)[nH]c1=O]

In one embodiment, the compound of formula (I) is not:

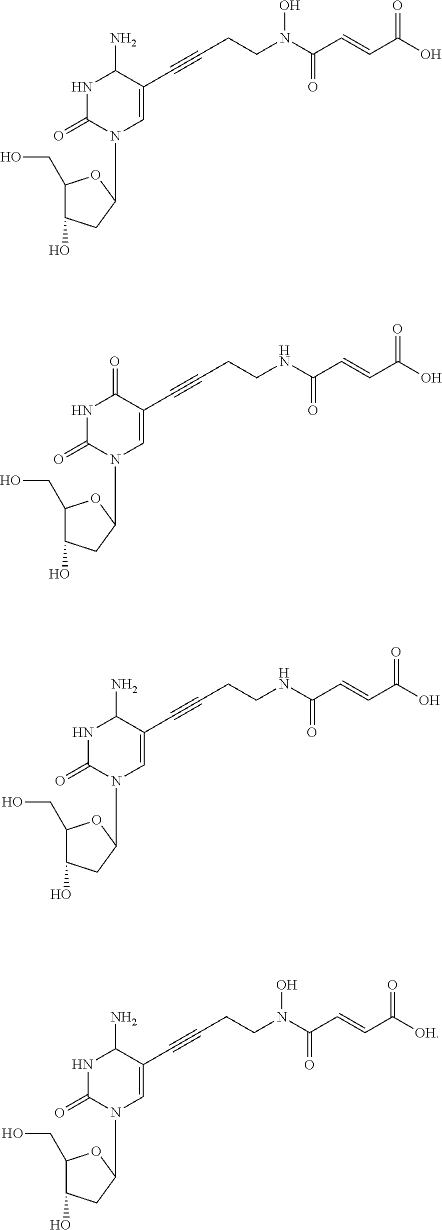 [SMILES: NC1NC(=O)N(C2C[C@H](O)C(CO)O2)C=C1C#CCCN(O)C(=O)/C=C/C(=O)O ; O=C(O)/C=C/C(=O)NCCC#Cc1cn(C2C[C@H](O)C(CO)O2)c(=O)[nH]c1=O ; NC1NC(=O)N(C2C[C@H](O)C(CO)O2)C=C1C#CCCNC(=O)/C=C/C(=O)O ; NC1NC(=O)N(C2C[C@H](O)C(CO)O2)C=C1C#CCCN(O)C(=O)/C=C/C(=O)O]

In one embodiment, the compound of formula (I) is not:

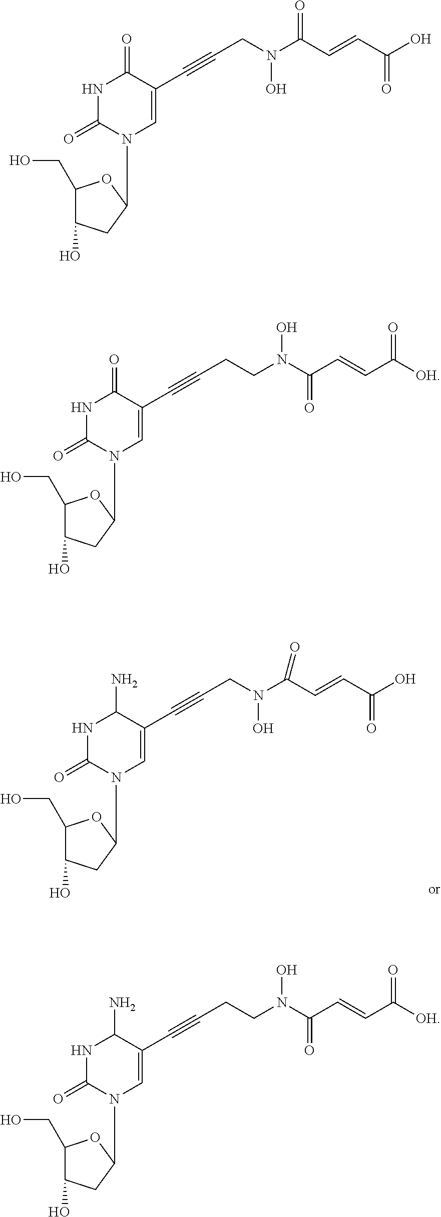 [SMILES: O=C(O)/C=C/C(=O)N(O)CC#Cc1cn(C2C[C@H](O)C(CO)O2)c(=O)[nH]c1=O ; O=C(O)/C=C/C(=O)N(O)CCC#Cc1cn(C2C[C@H](O)C(CO)O2)c(=O)[nH]c1=O ; NC1NC(=O)N(C2C[C@H](O)C(CO)O2)C=C1C#CCN(O)C(=O)/C=C/C(=O)O ; NC1NC(=O)N(C2C[C@H](O)C(CO)O2)C=C1C#CCCN(O)C(=O)/C=C/C(=O)O]

In one embodiment, the compound of formula (I) is not:

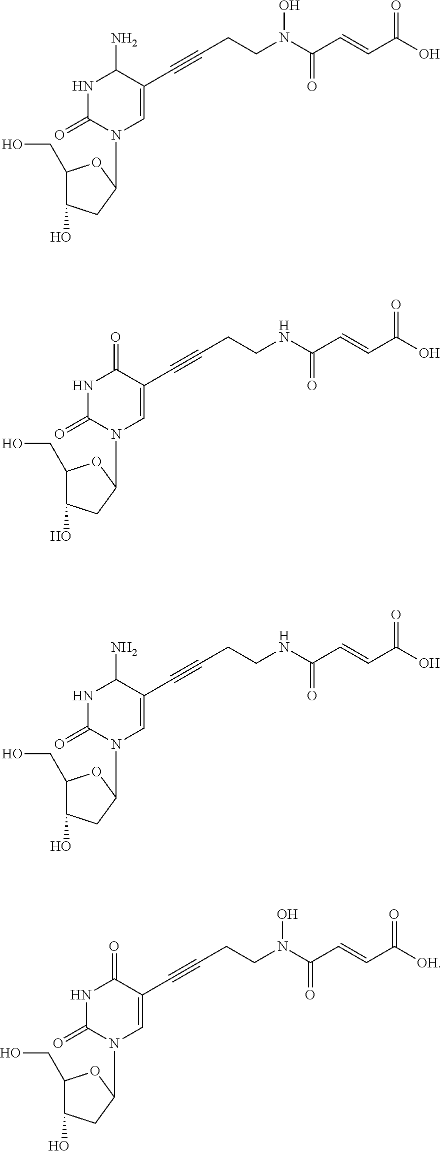 [SMILES: NC1NC(=O)N(C2C[C@H](O)C(CO)O2)C=C1C#CCCN(O)C(=O)/C=C/C(=O)O ; O=C(O)/C=C/C(=O)NCCC#Cc1cn(C2C[C@H](O)C(CO)O2)c(=O)[nH]c1=O ; NC1NC(=O)N(C2C[C@H](O)C(CO)O2)C=C1C#CCCNC(=O)/C=C/C(=O)O ; O=C(O)/C=C/C(=O)N(O)CCC#Cc1cn(C2C[C@H](O)C(CO)O2)c(=O)[nH]c1=O]

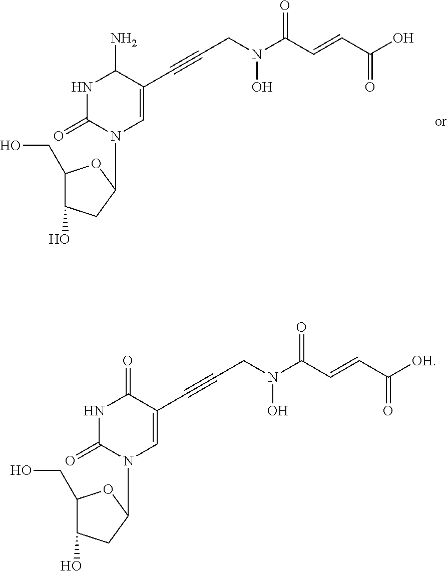 [SMILES: NC1NC(=O)N(C2C[C@H](O)C(CO)O2)C=C1C#CCN(O)C(=O)/C=C/C(=O)O ; O=C(O)/C=C/C(=O)N(O)CC#Cc1cn(C2C[C@H](O)C(CO)O2)c(=O)[nH]c1=O]

Processes for preparing compounds of formula (I) are provided as further embodiments of the invention and are illustrated by the procedures.

In cases where compounds are sufficiently basic or acidic, a salt of a compound of formula (I) can be useful as an intermediate for isolating or purifying a compound of formula (I). Additionally, administration of a compound of formula (I) as a pharmaceutically acceptable acid or base salt may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts.

Salts may be obtained using standard procedures well known in organic synthesis, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

The compounds of formula (I) can be formulated as pharmaceutical compositions and administered to a mammalian host, such as a human patient in a variety of forms adapted to the chosen route of administration, i.e., orally or parenterally, by intravenous, intramuscular, topical or subcutaneous routes.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds of formula (I) to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compounds of formula (I) can be determined by comparing their in vitro activity, and in vivo activity in animal models. Methods for the extrapolation of effective dosages in mice, and other animals, to humans are known to the art; for example, see U.S. Pat. No. 4,938,949.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

Compounds of the invention can also be administered in combination with other therapeutic agents, for example, other agents that are useful for the treatment of cancer. Accordingly, in one embodiment the invention also provides a composition comprising a compound of formula (I), or a pharmaceutically acceptable salt thereof, at least one other therapeutic agent, and a pharmaceutically acceptable diluent or carrier. The invention also provides a kit comprising a compound of formula (I), or a pharmaceutically acceptable salt thereof, at least one other therapeutic agent, packaging material, and instructions for administering the compound of formula (I) or the pharmaceutically acceptable salt thereof and the other therapeutic agent or agents to an animal to treat cancer.

Compounds of the invention can also be administered in combination with other therapeutic agents, for example, other agents that are useful for the treatment of eating disorders (e.g. anorexia). Accordingly, in one embodiment the invention also provides a composition comprising a compound of formula (I), or a pharmaceutically acceptable salt thereof, at least one other therapeutic agent, and a pharmaceutically acceptable diluent or carrier. The invention also provides a kit comprising a compound of formula (I), or a pharmaceutically acceptable salt thereof, at least one other therapeutic agent, packaging material, and instructions for administering the compound of formula (I) or the pharmaceutically acceptable salt thereof and the other therapeutic agent or agents to an animal to treat an eating disorder.

Compounds of the invention can also be used as adjuvants in CAR-T cell immunotherapy by inducing T cell differentiation to long living memory cells. Accordingly, in one embodiment the invention also provides a composition comprising a compound of formula (I), or a pharmaceutically acceptable salt thereof, at least one other therapeutic agent, and a pharmaceutically acceptable diluent or carrier. The invention also provides a kit comprising a compound of formula (I), or a pharmaceutically acceptable salt thereof, at least one other therapeutic agent, packaging material, and instructions for administering the compound of formula (I) or the pharmaceutically acceptable salt thereof and the other therapeutic agent or agents to patient cells to treat cancer via immunotherapy.

The invention will now be illustrated by the following non-limiting Examples.

### EXAMPLES

Chemical reactions were conducted with oven-dried glassware under argon atmosphere unless otherwise stated. Thin-layer chromatography (TLC) was performed with Analtech silica uniplates and visualized under 254 nm UV light or with KMnO₄ staining. Column chromatography was conducted with 60 mesh silica gel. NMR spectra were acquired on a 500 MHz Bruker spectrometer at room temperature. MS spectra were acquired on a Thermo Scientific LTQ XL Ion Trap.

### Example 1. Chemical Preparations

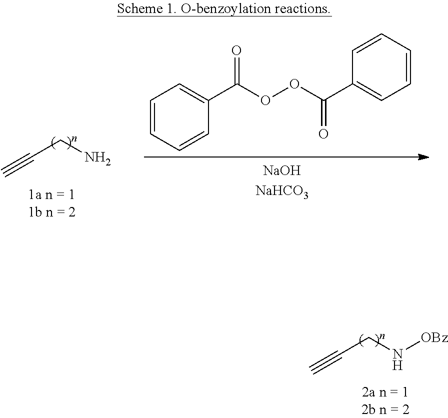

General Method for O-benzoylation Reactions (2a-b):

To a round-bottomed flask were sequentially added DCM (120 mL), starting alkyne 1a or 1b (60 mmol, 1 equiv), and NaHCO₃/NaOH aqueous solution (pH 10.5, 60 mL). After five minutes of vigorous stirring, benzoyl peroxide was added (72 mmol, 1.2 equiv) and the reaction mixture was allowed to stir for four hours at room temperature. The organic layer was collected, then the aqueous layer was extracted with DCM (6×20 mL). The organic layers were pooled, washed with brine, dried with magnesium sulfate, and concentrated in vacuo. Purification via silica gel flash column chromatography (2 column volumes of 0%, 5 columns volumes from 0% to 10%, 6 column volumes of 10%, 5 column volumes from 10% to 20%, and 8 column volumes of 20% EtOAc in hexanes) afforded compound 2a-b.

### Preparation of O-benzoyl-N-(prop-2-yn-1-yl) hydroxylamine (2a)

Orange oil (32% yield). R_{f}=0.32 (20% EtOAc in hexanes). ¹H NMR (500 MHZ, CDCl₃) δ 2.26 (t, J=2.4 Hz, 1H), 3.92 (d, J=2.5 Hz, 2H), 7.46 (t, J=7.8 Hz, 2H), 7.56-7.62 (m, 1H), 8.03-8.07 (m, 2H). ¹³C NMR (126 MHz, CDCl₃) δ 41.76, 73.25, 78.58, 128.10, 128.68, 129.57, 133.63, 166.73. MS (ESI-TOF) m/z [M+H]⁺ calculated for C₁₀H₁₀NO₂⁺ 176.1, found 176.1.

### Preparation of O-benzoyl-N-(but-3-yn-1-yl) hydroxylamine (2b)

Clear, colorless oil (47% yield). R_{f}=0.32 (20% EtOAc in hexanes). ¹H NMR (500 MHz, CDCl₃) δ 2.05 (t, J=2.7 Hz, OH), 2.53 (td, J=6.7, 2.7 Hz, 1H), 3.30 (t, J=6.7 Hz, 1H), 7.42 (dd, J=8.7, 7.0 Hz, 1H), 7.51-7.59 (m, 1H), 7.99 (dd, J=8.4, 1.5 Hz, 1H). ¹³C NMR (126 MHz, CDCl₃) δ 17.53, 50.78, 70.36, 81.01, 128.25, 128.55, 129.36, 133.41, 166.47. MS (ESI-TOF) m/z [M+H]⁺ calculated for C₁₁H₁₂NO₂⁺ 190.1, found 190.1.

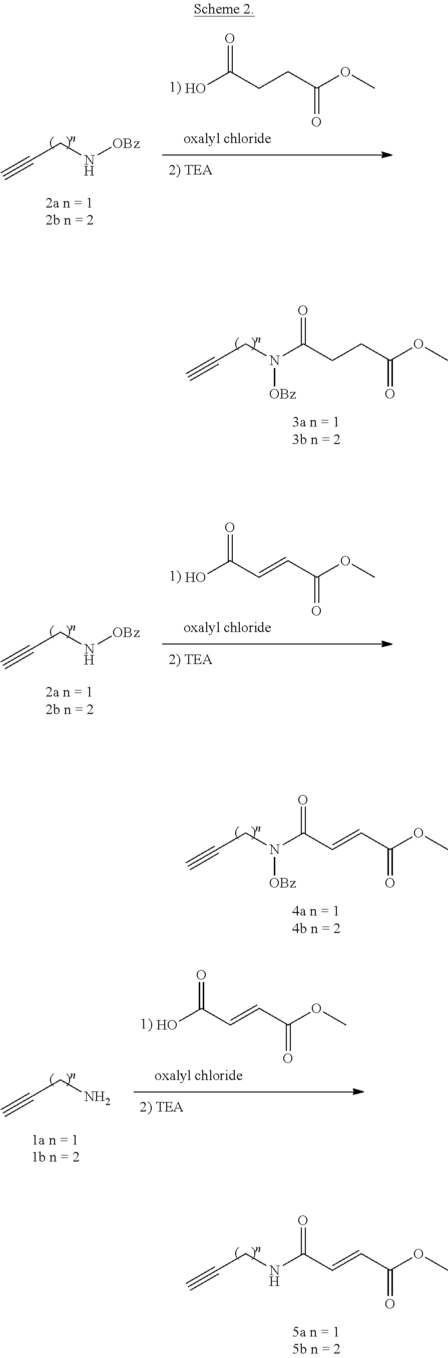

General Method for N-acylation Reactions (3a-d):

A round-bottomed flask was purged with argon and sequentially added were monomethyl succinate or monomethyl fumarate (1-1.8 equiv) and anhydrous DCM. After five minutes of stirring, oxalyl chloride (1.05-1.85 equiv) was added dropwise, followed by anhydrous DMF (3-8 drops). The acid chloride reaction mixture was allowed to stir for two hours at room temperature. After two hours, a second round-bottomed flask was placed in an ice bath, purged with argon and were sequentially added compound 2a-b or 1a-b (1 equiv), anhydrous THF, and triethylamine (1.85 equiv). After five minutes of stirring, the acid chloride reaction mixture was transferred to the second reaction mixture via anhydrous transfer and was added dropwise. The reaction mixture was allowed to stir for 18 hours at room temperature. Afterwards, DCM (10 mL) and water (10 mL) was added to the reaction mixture. The organic layer was collected, then the aqueous layer was extracted with DCM (4×10 mL). The organic layers were pooled, washed with brine, dried with magnesium sulfate, and concentrated in vacuo. Purification via silica gel flash column chromatography (2 column volumes of 0%, 5 columns volumes from 0% to 15%, 4 column volumes of 15%, 5 column volumes from 15% to 20%, and 8 column volumes of 20% EtOAc in hexanes) afforded compound 3a-b, 4a-b, and 5a-b.

### Preparation of methyl 4-((benzoyloxy)(prop-2-yn-1-yl)amino)-4-oxobutanoate (3a)

Cloudy, colorless oil (57% yield). R_{f}=0.33 (40% EtOAc in hexanes). ¹H NMR (500 MHz, CDCl₃) δ 2.25 (t, J=2.5 Hz, 1H), 2.64-2.72 (m, 4H), 3.68 (s, 3H), 4.61 (d, J=2.5 Hz, 2H), 7.50-7.56 (m, 2H), 7.67-7.72 (m, 1H), 8.13 (dd, J=8.4, 1.4 Hz, 2H). ¹³C NMR (126 MHz, CDCl₃) δ 27.68, 28.32, 38.14, 52.02, 73.06, 76.59, 126.60, 129.09, 130.34, 134.79, 164.36, 172.99. MS (ESI-TOF) m/z [M+H]⁺ calculated for C₁₅H₁₆NO₅⁺ 290.1, found 290.1.

### Preparation of methyl 4-((benzoyloxy)(but-3-yn-1-yl)amino)-4-oxobutanoate (3b)

Clear, colorless oil (88% yield). R_{f}=0.45 (40% EtOAc in hexanes). ¹H NMR (500 MHz, CDCl₃) δ 1.96 (s, 1H), 2.58 (td, J=7.1, 2.7 Hz, 2H), 2.65 (s, 4H), 3.67 (s, 3H), 4.00 (t, J=7.1 Hz, 2H), 7.52 (t, J=7.7 Hz, 2H), 7.68 (t, J=7.5 Hz, 1H), 8.06-8.14 (m, 2H). ¹³C NMR (126 MHz, CDCl₃) δ 17.46, 27.37, 28.40, 47.28, 51.95, 70.28, 80.76, 126.68, 129.05, 130.25, 134.74, 164.46, 172.21, 173.10. (ESI-TOF) m/z [M+H]⁺ calculated for C₁₆H₁₇NO₅⁺ 304.1, found 304.1.

### Preparation of methyl (E)-4-(but-3-yn-1-ylamino)-4-oxobut-2-enoate (4a)

White solid (93% yield). R_{f}=0.42 (40% EtOAc in hexanes). ¹H NMR (CHLOROFORM-d, 400 MHz): d=8.13 (dd, J=8.2, 1.2 Hz, 2H), 7.71 (s, 1H), 7.50-7.63 (m, 2H), 7.16 (d, J=15.7 Hz, 1H), 6.98 (d, J=15.3 Hz, 1H), 4.69 (d, J=2.3 Hz, 2H), 3.76 ppm (s, 3H) MS (ESI-TOF) m/z [M+H]⁺ calculated for C₁₅H₁₄NO₅⁺ 288.1, found 288.1.

### Preparation of methyl (E)-4-((benzoyloxy)(but-3-yn-1-yl)amino)-4-oxobut-2-enoate (4b)

White solid (80% yield). R_{f}=0.35 (40% EtOAc in hexanes). ¹H NMR (METHANOL-d₄, 400 MHz): d=8.02-8.19 (m, 2H), 7.75 (t, J=7.4 Hz, 1H), 7.59 (t, J=7.6 Hz, 2H), 7.19 (br. s., 1H), 6.84 (d, J=15.7 Hz, 1H), 4.06 (br. s., 2H), 3.74 (br. s., 3H), 2.62 ppm (td, J=6.6, 2.5 Hz, 2H). (ESI-TOF) m/z [M+H]⁺ calculated for C₁₆H₁₀NO₃⁺ 302.1, found 302.1.

### Preparation of methyl (E)-4-oxo-4-(prop-2-yn-1-ylamino)but-2-enoate (5a)

White solid (97% yield). R_{f}=0.20 (40% EtOAc in hexanes). ¹H NMR (500 MHZ, CDCl₃) δ 6.94 (d, J=15.4 Hz, 1H), 6.65 (d, J=15.4 Hz, 1H), 4.17 (s, 2H), 3.80 (s, 3H), 2.27 (s, 1H). (ESI-TOF) m/z [M+H]⁺ calculated for C₈H₁₀NO₃⁺ 168.1, found 168.1.

### Preparation of methyl (E)-4-(but-3-yn-1-ylamino)-4-oxobut-2-enoate (5b)

White solid (95% yield). R_{f}=0.15 (40% EtOAc in hexanes). ¹H NMR (500 MHZ, CDCl₃) δ 6.96 (d, J=15.4 Hz, 1H), 6.81 (d, J=15.4 Hz, 1H), 6.66 (d, J=7.7 Hz, 1H), 3.77 (s, 3H), 3.49 (q, J=6.3 Hz, 2H), 2.44 (td, J=6.4, 2.6 Hz, 2H), 2.01 (t, J=2.6 Hz, 1H). ¹³C NMR (126 MHZ, CDCl₃) δ 19.2886, 38.5210, 52.3043, 70.3936, 81.2661, 130.1677, 136.5433, 163.8207, 166.1825. (ESI-TOF) m/z [M+H]⁺ calculated for C₁₀H₁₂NO₃⁺ 182.1, found 182.1.

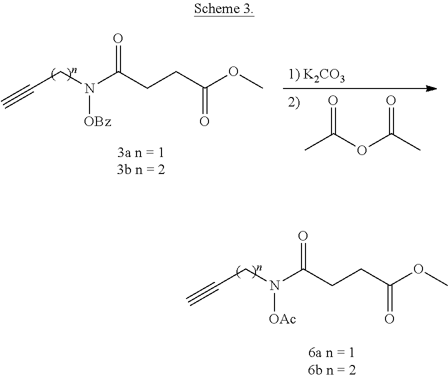

General Method for Consecutive Debenzoylation-Acetylation Reactions (6a-b):

A round-bottomed flask was purged with argon and sequentially added were compound 3a-b (1 equiv), anhydrous methanol, and potassium carbonate (1.2 equiv). The reaction mixture was allowed to stir for 30 minutes at room temperature and was monitored by TLC (KMnO₄ stain). Afterwards, the methanol was removed in vacuo and to the reaction mixture was added ethyl acetate (20 mL) and water (20 mL). The reaction mixture was brought to a pH of 12 by adding NaOH (1 M) in which the aqueous layer was collected. Subsequently, the aqueous layer was acidified to a pH of 4 by adding HCl (1 M). The aqueous phase was extracted with ethyl acetate (3×15 mL). The organic layers were pooled, washed with brine, dried with magnesium sulfate, and concentrated in vacuo to afford a crude oil which was used subsequently without additional purification. To another round-bottomed flask were sequentially added the crude oil (1 equiv), triethylamine (10 equiv), acetic anhydride (10 equiv), and ethyl acetate (minimal amount required to dissolve the crude oil into the reaction mixture). The reaction mixture was allowed to stir for 30 minutes at room temperature and was monitored by TLC (KMnO₄ stain). Afterwards, water (10 mL) was added to the reaction mixture. The organic layer was collected, then the aqueous layer was extracted with ethyl acetate (3×10 mL). The organic layers were pooled, neutralized with saturated NaHCO₃ solution, washed with brine, dried with magnesium sulfate, and concentrated in vacuo to afford compound 6a-b.

### Preparation of methyl 4-(acetoxy(prop-2-yn-1-yl)amino)-4-oxobutanoate (6a)

Orange oil (52% yield). R_{f}=0.42 (50% EtOAc in hexanes, KMnO₄ stain). ¹H NMR (500 MHz, CDCl₃) δ 2.22 (s, 3H), 2.25 (t, J=2.5 Hz, 1H), 2.60 (dq, J=10.6, 5.9 Hz, 4H), 3.65 (s, 3H), 4.45 (d, J=2.6 Hz, 2H). ¹³C NMR (126 MHz, CDCl₃) δ 18.37, 27.44, 28.19, 37.91, 51.91, 72.95, 76.40, 168.21, 172.18, 172.84. (ESI-TOF) m/z [M+H]⁺ calculated for C₁₀H₁₄NO₅⁺ 228.1, found 228.1.

### Preparation of methyl 4-(acetoxy(but-3-yn-1-yl)amino)-4-oxobutanoate (6b)

Orange oil (36% yield). R_{f}=0.37 (50% EtOAc in hexanes, KMnO₄ stain). ¹H NMR (500 MHz, CDCl₃) δ 1.98 (t, J=2.8 Hz, 1H), 2.19 (s, 3H), 2.41-2.50 (m, 2H), 2.50-2.64 (m, 4H), 3.64 (s, 3H), 3.83 (t, J=7.1 Hz, 2H). ¹³C NMR (126 MHz, CDCl₃) δ 17.25, 18.49, 27.17, 28.32, 46.96, 51.86, 70.15, 80.67, 168.36, 171.80, 172.96. (ESI-TOF) m/z [M+H]⁺ calculated for C₁₁H₁₆NO₅⁺ 242.1, found 242.1.

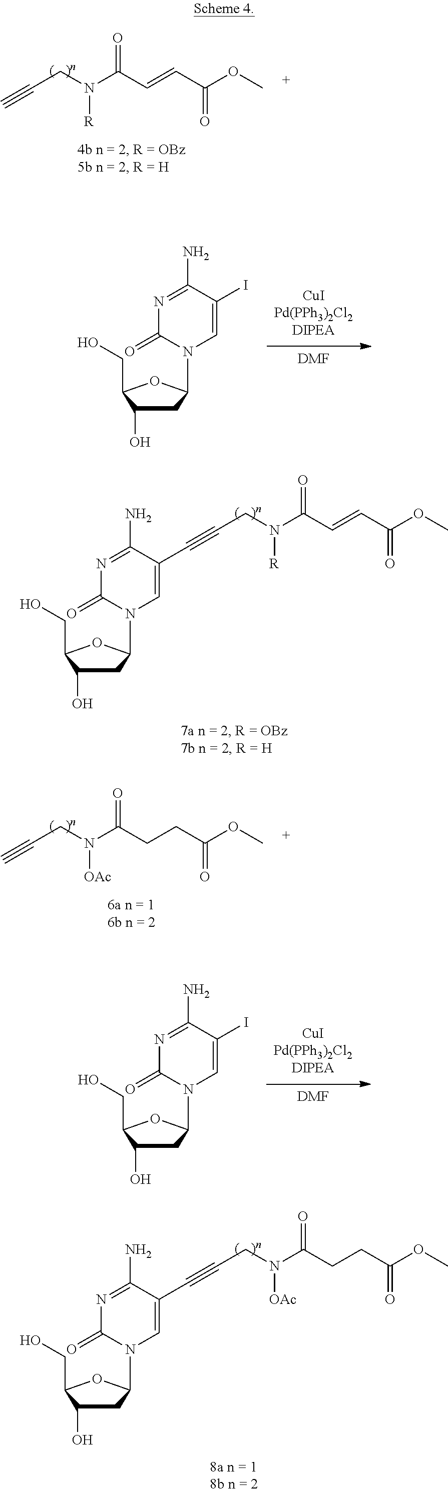

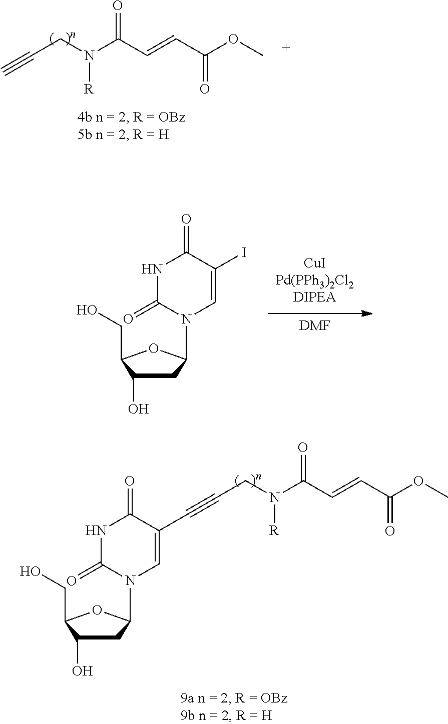

### General Method for Sonogashira Cross Coupling Reaction

A Schlenk flask was purged with argon and sequentially added were 5-iodo-2′-deoxycytidine or 5-Iodo-2′-deoxyuridine (1 equiv), Pd(PPh₃)₂Cl₂ (0.1 equiv), CuI (0.05 equiv), half of the anhydrous DMF (1 mL), and anhydrous N,N-diisopropylethylamine (2 equiv). A separate round-bottomed flask was purged with argon and sequentially added compound 4b, 5b, or 6a-b. (1.5 equiv) and half of the anhydrous DMF (1 mL). After allowing each reaction mixture to stir and purge for thirty minutes, the reaction mixture in the round-bottomed flask was transferred to the reaction mixture in the Schlenk flask via anhydrous transfer and was added dropwise. Afterwards, the reaction was heated to 50 C.° and was covered in aluminum foil to achieve dark conditions, and the reaction mixture was allowed to stir for 24-91 hours at these conditions, as tracked by TLC. The reaction mixture was then concentrated in vacuo to afford a dark crude oil. Purification via silica gel flash column chromatography (2 column volumes of 0%, 5 column volumes from 0% to 10%, and 20 column volumes of 10% methanol in DCM) afforded compounds 7a-b, 8a-b, and 9a-b.

### Preparation of methyl (E)-4-((4-(4-amino-1-((2R,4S,5R)-4-hydroxy-5-(hydroxymethyl)-tetrahydrofuran-2-yl)-2-oxo-1,2-dihydropyrimidin-5-yl)but-3-yn-1-yl) (benzoyloxy)amino)-4-oxobut-2-enoate (7a)

Light yellow foam (35% yield). ¹H NMR (500 MHZ, MeOD) δ=8.07 (s, 1H), 7.97-8.05 (m, 2H), 7.61-7.67 (m, 1H), 7.46 (t, J=7.8 Hz, 2H), 6.75 (d, J=15.3 Hz, 1H), 6.07 (t, J=6.3 Hz, 1H), 4.22-4.28 (m, 1H), 4.09 (br. s., 2H), 3.84 (q, J=3.4 Hz, 1H), 3.66-3.75 (m, 2H), 3.57-3.66 (m, 3H), 2.76 (t, J=6.1 Hz, 1H), 2.24-2.31 (m, 1H), 2.00 ppm (dt, J=13.4, 6.6 Hz, 1H). (ESI-TOF) m/z [M+H]⁺ calculated for C₂₅H₂₇N₄O₉⁺ 527.2, found 527.2.

### Preparation of methyl (E)-4-((4-(4-amino-1-((2R,4S,5R)-4-hydroxy-5-(hydroxymethyl)-tetrahydrofuran-2-yl)-2-oxo-1,2-dihydropyrimidin-5-yl)but-3-yn-1-yl)amino)-4-oxobut-2-enoate (7b)

This compound was taken forward to the following step crude (crude yield 56%).

### Preparation of methyl 4-(acetoxy(3-(4-amino-1-((2R,4S,5R)-4-hydroxy-5-(hydroxymethyl) tetrahydrofuran-2-yl)-2-oxo-1,2-dihydropyrimidin-5-yl) prop-2-yn-1-yl)amino)-4-oxobutanoate (8a)

Light orange solid (10% yield). R_{f}=0.23 (15% methanol in DCM). ¹H NMR (500 MHZ, MeOD) δ 2.14 (dt, J=13.2, 6.4 Hz, 1H), 2.27 (s, 3H), 2.39 (ddd, J=13.6, 6.2, 4.1 Hz, 1H), 2.59-2.70 (m, 4H), 3.67 (s, 3H), 3.74 (dd, J=12.1, 3.7 Hz, 1H), 3.82 (dd, J=12.1, 3.2 Hz, 1H), 3.95 (q, J=3.5 Hz, 1H), 4.36 (dt, J=6.3, 4.0 Hz, 1H), 4.72 (s, 2H), 6.19 (t, J=6.3 Hz, 1H), 8.35 (s, 1H). ¹³C NMR (126 MHZ, MeOD) δ 18.17, 28.27, 29.04, 42.40, 52.33, 62.43, 71.67, 88.01, 89.09, 89.58, 91.75, 146.35, 156.62, 166.34, 170.12, 174.69. (ESI-TOF) m/z [M+H]⁺ calculated for C₁₉H₂₅N₄O₉ 453.2, found 453.2.

### Preparation of methyl 4-(acetoxy(4-(4-amino-1-((2R,4S,5R)-4-hydroxy-5-(hydroxymethyl)-tetrahydrofuran-2-yl)-2-oxo-1,2-dihydropyrimidin-5-yl)but-3-yn-1-yl)amino)-4-oxobutanoate (8b)

Light yellow foam (42% yield). R_{f}=0.37 (10% methanol in DCM). ¹H NMR (500 MHZ, MeOD) δ 2.14 (td, J=13.5, 6.9 Hz, 1H), 2.25 (s, 3H), 2.38 (ddd, J=13.5, 6.2, 4.0 Hz, 1H), 2.55-2.70 (m, 4H), 2.75 (t, J=6.4 Hz, 2H), 3.64 (s, 3H), 3.73 (dd, J=12.0, 3.6 Hz, 1H), 3.82 (dd, J=12.0, 3.2 Hz, 1H), 3.92-3.98 (m, 3H), 4.36 (dt, J=6.3, 3.9 Hz, 1H), 6.20 (t, J=6.3 Hz, 1H), 8.26 (s, 1H). ¹³C NMR (126 MHZ, MeOD) δ 18.21, 19.00, 28.13, 29.14, 42.35, 52.28, 54.81, 62.52, 71.77, 87.95, 89.05, 93.77, 145.39, 156.72, 166.58, 174.64. (ESI-TOF) m/z [M+H]⁺ calculated for C₂₀H₂₇N₄O₉⁺ 467.2, found 467.2.

### Preparation of methyl (E)-4-((benzoyloxy)(4-(1-((2R,4S,5R)-4-hydroxy-5-(hydroxymethyl)-tetrahydrofuran-2-yl)-2,4-dioxo-1,2,3,4-tetrahydropyrimidin-5-yl)but-3-yn-1-yl)amino)-4-oxobut-2-enoate (9a)

¹H NMR (METHANOL-d₄, 400 MHz): d=8.05-8.20 (m, 3H), 7.69-7.75 (m, 1H), 7.55 (t, J=7.8 Hz, 2H), 6.83 (d, J=15.7 Hz, 1H), 6.20 (t, J=6.5 Hz, 1H), 4.38 (dt, J=6.2, 3.4 Hz, 1H), 4.15 (br. s., 2H), 3.93 (q, J=3.1 Hz, 1H), 3.68-3.83 (m, 5H), 2.82 (t, J=6.3 Hz, 2H), 2.24-2.33 (m, 1H), 2.12-2.23 ppm (m, 1H). (ESI-TOF) m/z [M+H]⁺ calculated for C₂₅H₂₆N₃O₁₀⁺ 528.2, found 528.2.

### Preparation of methyl (E)-4-((4-(1-((2R,4S,5R)-4-hydroxy-5-(hydroxymethyl)tetrahydrofuran-2-yl)-2,4-dioxo-1,2,3,4-tetrahydropyrimidin-5-yl)but-3-yn-1-yl)amino)-4-oxobut-2-enoate (9b)

This compound was taken forward to the following step crude (crude yield 43%).

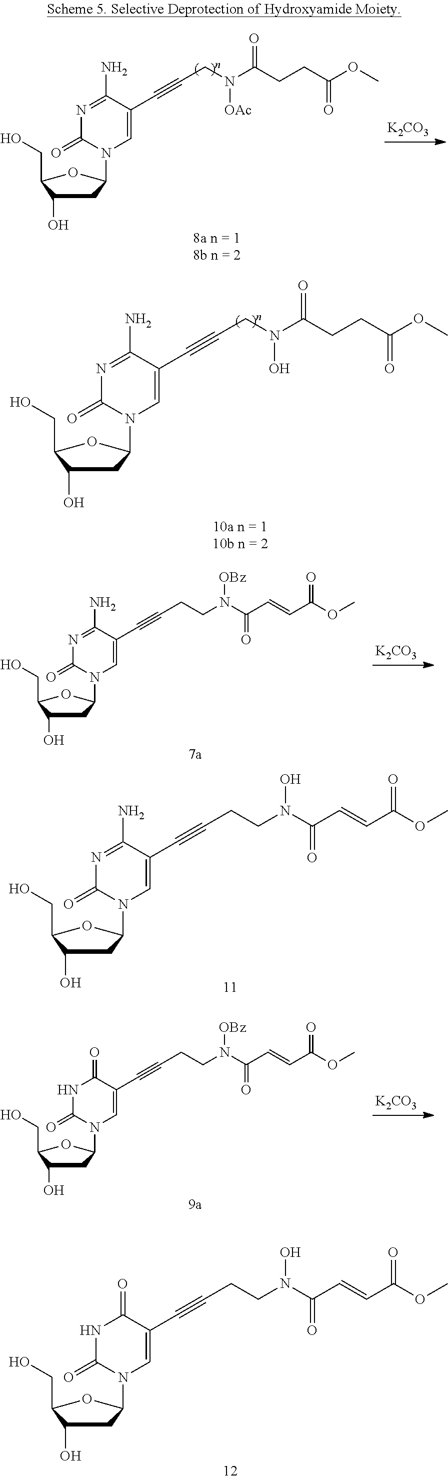

### General Method for Hydroxyamide Deprotection

A round-bottomed flask was purged with argon and sequentially added were compound 7a, 8a-b, or 9a (1 equiv), anhydrous methanol, and potassium carbonate (1.2 equiv). The reaction mixture was allowed to stir for 30 minutes at room temperature and was monitored by TLC (KMnO₄ stain). The reaction mixture was then concentrated in vacuo to afford a crude oil. Purification via silica gel flash column chromatography (1 column volume of 0%, 3 column volumes from 0% to 15%, and 30 column volumes of 15% methanol in DCM) and HPLC purification using a Sunfire C18 column (5 μm, 150×2.0 mm, 10.0 mL/min flow rate, buffer A: H₂O), buffer B: ACN) involved a gradient of 0-30% B (0-30 min), of 30-95% B (30-40 min), followed by an isocratic hold at 95% B (40-45 min) afforded compounds 10a-b, 11, or 12.

### Preparation of methyl 4-((3-(4-amino-1-((2R,4S,5R)-4-hydroxy-5-(hydroxymethyl)-tetrahydrofuran-2-yl)-2-oxo-1,2-dihydropyrimidin-5-yl) prop-2-yn-1-yl)(hydroxy)amino)-4-oxobutanoate (10a)

Light yellow solid (61% yield). R_{f}=0.24 (15% methanol in DCM, KMnO₄ stain). ¹H NMR (500 MHZ, MeOD) δ 2.14 (dt, J=13.3, 6.4 Hz, 1H), 2.39 (ddd, J=13.6, 6.2, 4.0 Hz, 1H), 2.61 (t, J=6.7 Hz, 2H), 2.82 (t, J=6.6 Hz, 2H), 3.67 (s, 3H), 3.74 (dd, J=12.0, 3.7 Hz, 1H), 3.82 (dd, J=12.1, 3.1 Hz, 1H), 3.95 (q, J=3.6 Hz, 1H), 4.36 (dt, J=7.2, 3.9 Hz, 1H), 4.60 (s, 2H), 6.20 (t, J=6.3 Hz, 1H), 8.33 (s, 1H). ¹³C NMR (126 MHz, MeOD) δ 28.39, 29.23, 40.15, 42.33, 49.85, 52.24, 62.47, 71.73, 75.36, 87.96, 89.04, 90.82, 92.12, 146.16, 156.68, 166.35, 175.08. (ESI-TOF) m/z [M−H]⁻ calculated for C₁₇H₂₁N₄O₈⁻ 409.1, found 409.1.

### Preparation of methyl 4-((4-(4-amino-1-((2R,4S,5R)-4-hydroxy-5-(hydroxymethyl)-tetrahydrofuran-2-yl)-2-oxo-1,2-dihydropyrimidin-5-yl)but-3-yn-1-yl)(hydroxy)amino)-4-oxobutanoate (10b)

White foam (59% yield). R_{f}=0.28 (15% methanol in DCM, KMnO₄ stain). 1H NMR (500 MHZ, MeOD) δ 2.13 (dt, J=13.3, 6.5 Hz, 1H), 2.37 (ddd, J=13.6, 6.2, 4.0 Hz, 1H), 2.59 (t, J=6.7 Hz, 2H), 2.78 (dt, J=34.3, 6.5 Hz, 4H), 3.64 (s, 3H), 3.70-3.88 (m, 4H), 3.94 (q, J=3.5 Hz, 1H), 4.36 (dt, J=6.3, 3.8 Hz, 1H), 6.20 (t, J=6.4 Hz, 1H), 8.24 (s, 1H). ¹³C NMR (126 MHZ, MeOD) δ 18.57, 28.32, 29.34, 42.35, 48.06, 52.21, 62.53, 71.79, 73.31, 87.91, 89.04, 93.13, 94.06, 145.22, 166.60, 174.99, 175.10. (ESI-TOF) m/z [M−H]⁻ calculated for C₁₈H₂₃N₄O₈⁻ 423.1, found 423.1.

### Preparation of methyl (E)-4-((4-(4-amino-1-((2R,4S,5R)-4-hydroxy-5-(hydroxymethyl)-tetrahydrofuran-2-yl)-2-oxo-1,2-dihydropyrimidin-5-yl)but-3-yn-1-yl)(hydroxy)amino)-4-oxobut-2-enoate (11)

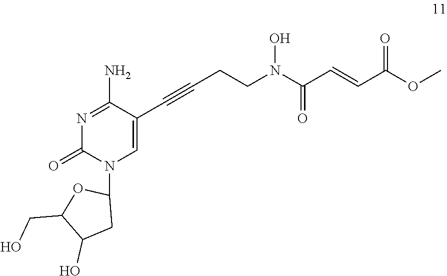 [SMILES: COC(=O)/C=C/C(=O)N(O)CCC#Cc1cn(C2CC(O)C(CO)O2)c(=O)nc1N]

Yellow solid (66% yield). R_{f}=0.48 (15% methanol in DCM, KMnO₄ stain). ¹H NMR (METHANOL-d₄, 500 MHz): d=8.13 (s, 1H), 7.57 (d, J=15.6 Hz, 1H), 6.68 (d, J=15.6 Hz, 1H), 6.09 (t, J=6.3 Hz, 1H), 4.22-4.30 (m, 1H), 3.81-3.93 (m, 3H), 3.66-3.74 (m, 4H), 3.57-3.66 (m, 2H), 3.21 (dt, J=3.3, 1.6 Hz, 2H), 2.71 (t, J=6.4 Hz, 2H), 2.23-2.32 (m, 1H), 1.98-2.06 ppm (m, 1H). (ESI-TOF) m/z [M−H]⁻; calculated for C₁₈H₂₁N₄O₈⁻ 421.1, found 421.1.

### Preparation of methyl (E)-4-(hydroxy (4-(1-((2R,4S,5R)-4-hydroxy-5-(hydroxymethyl)-tetrahydrofuran-2-yl)-2,4-dioxo-1,2,3,4-tetrahydropyrimidin-5-yl)but-3-yn-1-yl)amino)-4-oxobut-2-enoate (12)

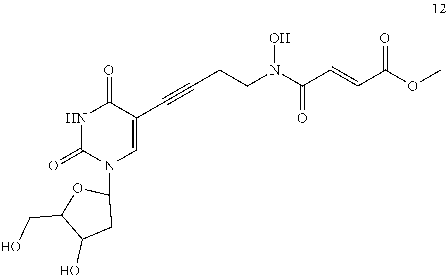 [SMILES: COC(=O)/C=C/C(=O)N(O)CCC#Cc1cn(C2CC(O)C(CO)O2)c(=O)[nH]c1=O]

Yellow solid (34% yield). R_{f}=0.32 (15% methanol in DCM, KMnO₄ stain). ¹H NMR (METHANOL-d₄, 400 MHz): d=8.22 (s, 1H), 7.67 (d, J=15.3 Hz, 1H), 6.73 (d, J=16.0 Hz, 1H), 6.22 (t, J=6.7 Hz, 1H), 4.36-4.42 (m, 1H), 3.86-3.96 (m, 2H), 3.68-3.85 (m, 3H), 2.75 (t, J=6.8 Hz, 2H), 2.28 (dd, J=6.1, 3.7 Hz, 1H), 2.14-2.24 ppm (m, 1H). (ESI-TOF) m/z [M−H]⁻ calculated for C₁₈H₂₀N₃O₉⁻ 422.1, found 422.1.

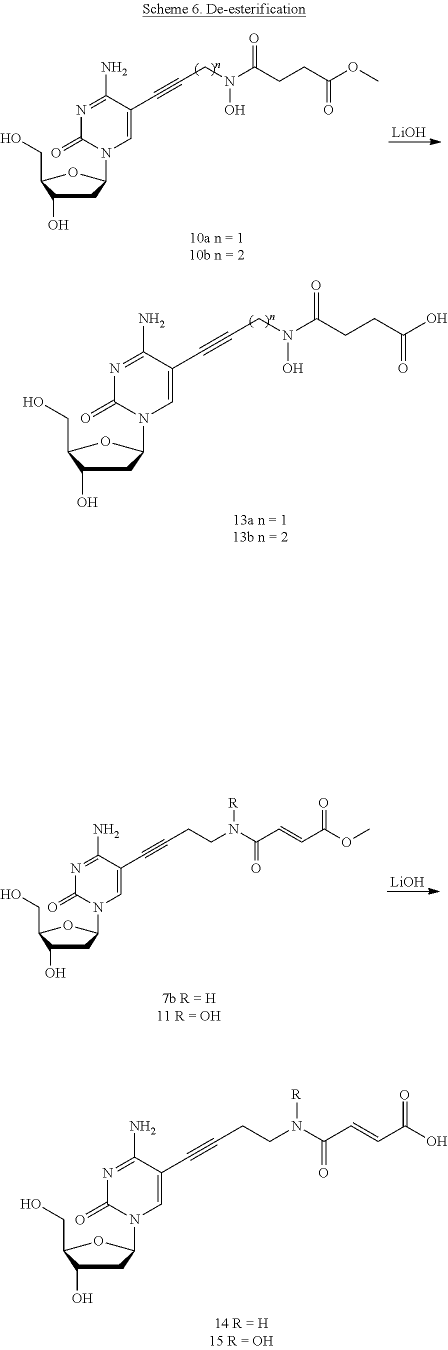

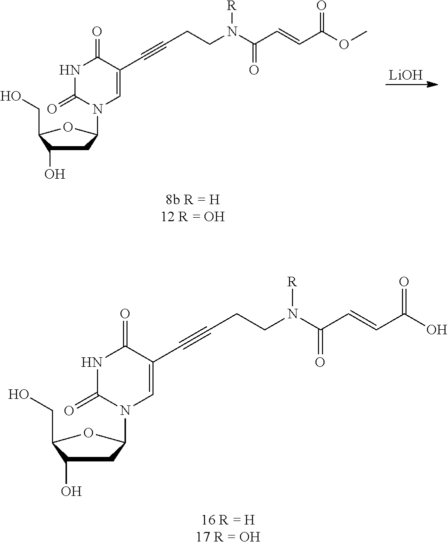

### General Method for Ester Hydrolysis

To a round-bottomed flask were sequentially added compounds 7b, 8b, 10a-b, 11, or 12 (1 equiv), THF, and water. After five minutes of stirring, lithium hydroxide (3 equiv) was added and the reaction mixture was allowed to stir for five minutes at room temperature. The reaction mixture was then concentrated in vacuo to afford a crude oil. Purification via silica gel flash column chromatography (1 column volume of 0%, 3 column volumes from 0% to 20%, 3 column volumes of 20%, 3 column volumes from 20% to 40%, and 20 column volumes of 40% methanol in DCM) and HPLC purification using a Sunfire C18 column (5 μm, 150×2.0 mm, 10.0 mL/min flow rate, buffer A: H₂O), buffer B: ACN) involved a gradient of 0-30% B (0-30 min), of 30-95% B (30-40 min), followed by an isocratic hold at 95% B (40-45 min) afforded compounds afforded compounds 13a-b, 14, 15, and 16.

### Preparation of 4-((3-(4-amino-1-((2R,4S,5R)-4-hydroxy-5-(hydroxymethyl)tetrahydrofuran-2-yl)-2-oxo-1,2-dihydropyrimidin-5-yl) prop-2-yn-1-yl)(hydroxy)amino)-4-oxobutanoic acid (13a)

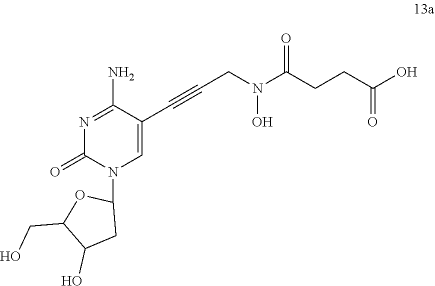 [SMILES: Nc1nc(=O)n(C2CC(O)C(CO)O2)cc1C#CCN(O)C(=O)CCC(=O)O]

Light yellow solid (56% yield). R_{f}=0.05 (40% methanol in DCM, KMnO₄ stain). ¹H NMR (500 MHZ, MeOD) δ 2.13 (dt, J=13.3, 6.5 Hz, 1H), 2.38 (ddd, J=13.6, 6.1, 4.0 Hz, 1H), 2.58 (t, J=6.8 Hz, 2H), 2.77 (t, J=6.7 Hz, 2H), 3.73 (dd, J=12.1, 3.7 Hz, 1H), 3.82 (dd, J=12.0, 3.1 Hz, 1H), 3.94 (q, J=3.5 Hz, 1H), 4.36 (dt, J=6.4, 3.9 Hz, 1H), 4.60 (s, 2H), 6.20 (t, J=6.3 Hz, 1H), 8.34 (s, 1H). ¹³C NMR (126 MHz, MeOD) δ 28.70, 30.30, 31.65, 36.95, 40.08, 42.40, 62.47, 71.73, 75.34, 87.96, 89.07, 90.78, 92.17, 143.84, 146.15, 156.71, 166.42, 175.49. (ESI-TOF) m/z [M−H]⁻ calculated for C₁₆H₁₉N₄O₈⁻ 395.1, found 395.1.

### Preparation of 4-((4-(4-amino-1-((2R,4S,5R)-4-hydroxy-5-(hydroxymethyl)tetrahydrofuran-2-yl)-2-oxo-1,2-dihydropyrimidin-5-yl)but-3-yn-1-yl)(hydroxy)amino)-4-oxobutanoic acid (13b)

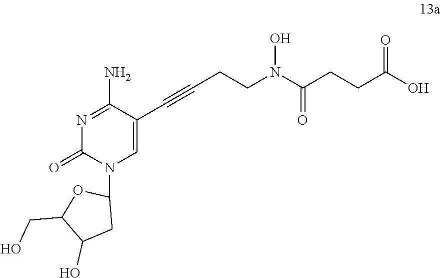 [SMILES: Nc1nc(=O)n(C2CC(O)C(CO)O2)cc1C#CCCN(O)C(=O)CCC(=O)O]

White solid (58% yield). R_{f}=0.05 (40% methanol in DCM, KMnO₄ stain). ¹H NMR (500 MHz, MeOD) δ 2.13 (dt, J=13.3, 6.5 Hz, 1H), 2.37 (ddd, J=13.5, 6.1, 4.0 Hz, 1H), 2.55 (t, J=7.0 Hz, 2H), 2.75 (q, J=6.6 Hz, 4H), 3.70-3.90 (m, 4H), 3.92-3.96 (m, 1H), 4.36 (dt, J=6.4, 3.9 Hz, 1H), 6.20 (t, J=6.3 Hz, 1H), 8.24 (s, 1H). ¹³C NMR (126 MHZ, MeOD) δ 18.58, 29.20, 32.01, 42.35, 47.87, 49.85, 62.50, 71.76, 73.23, 87.86, 89.03, 93.19, 94.16, 145.21, 156.82, 166.58, 175.65. (ESI-TOF) m/z [M−H]⁻ calculated for C₁₇H₂₁N₄O₈⁻ 409.1, found 409.1.

### Preparation of (E)-4-((4-(4-amino-1-((2R,4S,5R)-4-hydroxy-5-(hydroxymethyl)tetrahydrofuran-2-yl)-2-oxo-1,2-dihydropyrimidin-5-yl)but-3-yn-1-yl)amino)-4-oxobut-2-enoic acid (14)

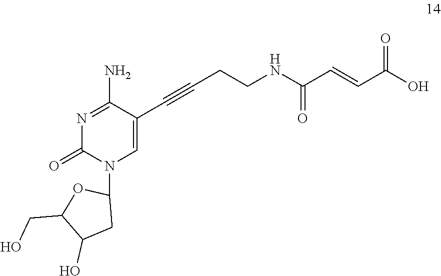 [SMILES: Nc1nc(=O)n(C2CC(O)C(CO)O2)cc1C#CCCNC(=O)/C=C/C(=O)O]

White solid (48% yield). R_{f}=0.04 (40% methanol in DCM, KMnO₄ stain). ¹H NMR (METHANOL-d₄, 500 MHz): d=8.15 (s, 1H), 6.77 (d, J=15.6 Hz, 1H), 6.64 (d, J=15.6 Hz, 1H), 6.14 (t, J=6.6 Hz, 1H), 4.28-4.32 (m, 1H), 3.83 (q, J=3.2 Hz, 1H), 3.71 (dd, J=12.2, 3.1 Hz, 1H), 3.64 (dd, J=12.1, 3.5 Hz, 1H), 3.37 (td, J=6.6, 2.6 Hz, 2H), 2.53 (t, J=6.7 Hz, 2H), 2.19 (dd, J=6.1, 3.7 Hz, 1H), 2.08-2.15 ppm (m, 1H). (ESI-TOF) m/z [M−H]⁻ calculated for C₁₇H₂₀N₄O₇⁻ 391.1, found 391.1.

### Preparation of (E)-4-((4-(4-amino-1-((2R,4S,5R)-4-hydroxy-5-(hydroxymethyl)tetrahydrofuran-2-yl)-2-oxo-1,2-dihydropyrimidin-5-yl)but-3-yn-1-yl)(hydroxy)amino)-4-oxobut-2-enoic acid (15)

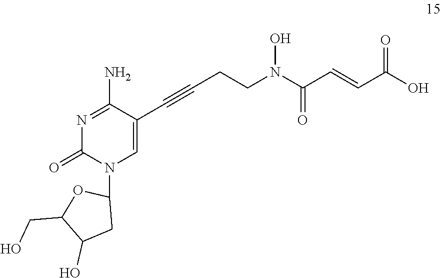 [SMILES: Nc1nc(=O)n(C2CC(O)C(CO)O2)cc1C#CCCN(O)C(=O)/C=C/C(=O)O]

White solid (41% yield). R_{f}=0.04 (40% methanol in DCM, KMnO₄ stain). ¹H NMR (METHANOL-d₄, 500 MHz): d=8.13 (s, 1H), 7.34 (d, J=15.9 Hz, 1H), 6.74 (d, J=15.6 Hz, 1H), 6.10 (t, J=6.3 Hz, 1H), 4.23-4.28 (m, 1H), 3.78-3.94 (m, 4H), 3.71 (dd, J=12.2, 2.7 Hz, 1H), 3.63 (dd, J=12.1, 3.5 Hz, 1H), 2.70 (t, J=6.3 Hz, 2H), 2.22-2.30 (m, 1H), 2.02 ppm (dt, J=13.4, 6.6 Hz, 1H). ¹³C NMR (METHANOL-d₄, 126 MHz): d=165.8, 163.4, 149.8, 143.5, 133.9, 99.3, 90.7, 87.7, 85.5, 72.5, 70.7, 61.2, 40.3, 38.2, 19.4 ppm. (ESI-TOF) m/z [M−H]⁻ calculated for C₁₇H₁₉N₄O₈⁻ 407.1, found 407.1.

### Preparation of (E)-4-((4-(1-((2R,4S,5R)-4-hydroxy-5-(hydroxymethyl)tetrahydrofuran-2-yl)-2,4-dioxo-1,2,3,4-tetrahydropyrimidin-5-yl)but-3-yn-1-yl)amino)-4-oxobut-2-enoic acid (16)

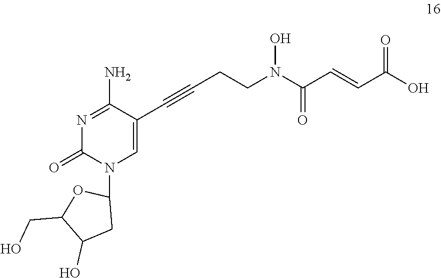 [SMILES: Nc1nc(=O)n(C2CC(O)C(CO)O2)cc1C#CCCN(O)C(=O)/C=C/C(=O)O]

White solid (40% yield). R_{f}=0.04 (40% methanol in DCM, KMnO₄ stain). ¹H NMR (METHANOL-d₄, 500 MHz): d=8.15 (s, 1H), 6.70 (d, J=15.6 Hz, 1H), 6.58 (d, J=15.6 Hz, 1H), 6.15 (t, J=6.7 Hz, 1H), 4.28-4.33 (m, 1H), 3.83 (q, J=3.2 Hz, 1H), 3.71 (dd, J=12.2, 3.1 Hz, 1H), 3.65 (dd, J=12.1, 3.5 Hz, 1H), 3.30-3.40 (m, 2H), 2.52 (t, J=6.7 Hz, 2H), 2.18 (dd, J=6.3, 3.5 Hz, 1H), 2.07-2.15 ppm (m, 1H). (ESI-TOF) m/z [M−H]⁻ calculated for C₁₇H₁₈N₃O₈⁻ 392.1, found 392.1.

### Preparation of (E)-4-(hydroxy (4-(1-((2R,4S,5R)-4-hydroxy-5-(hydroxymethyl)tetrahydrofuran-2-yl)-2,4-dioxo-1,2,3,4-tetrahydropyrimidin-5-yl)but-3-yn-1-yl)amino)-4-oxobut-2-enoic acid (17)

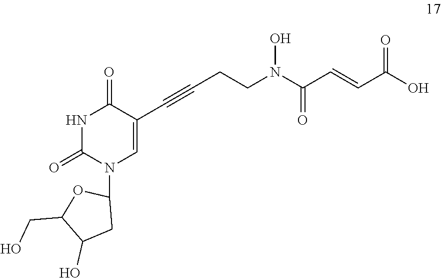 [SMILES: O=C(O)/C=C/C(=O)N(O)CCC#Cc1cn(C2CC(O)C(CO)O2)c(=O)[nH]c1=O]

¹H NMR (METHANOL-d₄, 400 MHz): d=8.21 (s, 1H), 7.34 (d, J=14.1 Hz, 1H), 6.85 (d, J=15.7 Hz, 1H), 6.23 (t, J=6.7 Hz, 1H), 4.40 (dt, J=6.1, 3.2 Hz, 1H), 3.87-3.97 (m, 2H), 3.70-3.86 (m, 3H), 2.74 (br. s., 2H), 2.25-2.32 (m, 1H), 2.15-2.25 ppm (m, 1H). (ESI-TOF) m/z [M−H]⁻ calculated for C₁₇H₁₈N₃O₉⁻ 408.1, found 408.1.

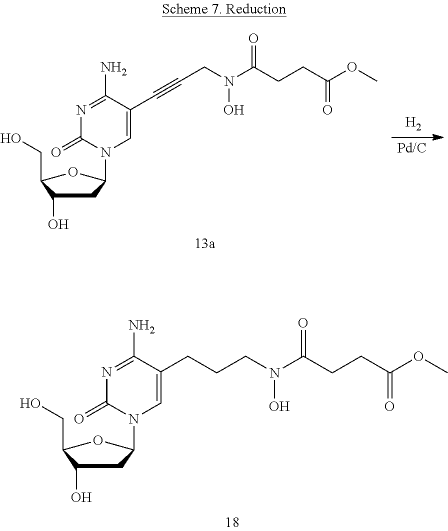

### General Method for Reduction

To a hydrogenation flask were added 13a (1 equiv), Pd/C (0.01 equiv) and MeOH (5 mL). The flask was purged with N₂ and was then placed under H₂ at a pressure of 45 bar on a hydrogenator. The reaction was shaken overnight. The reaction was then filtered over celite and concentrated in vacuo and subjected to HPLC purification using a Sunfire C18 column (5 μm, 150×2.0 mm, 10.0 mL/min flow rate, buffer A: H₂O), buffer B: ACN) involved a gradient of 0-30% B (0-30 min), of 30-95% B (30-40 min), followed by an isocratic hold at 95% B (40-45 min) to afford compound 18.

### Preparation of 4-((3-(4-amino-1-((2R,4S,5R)-4-hydroxy-5-(hydroxymethyl)tetrahydrofuran-2-yl)-2-oxo-1,2-dihydropyrimidin-5-yl) propyl)(hydroxy)amino)-4-oxobutanoic acid (18)

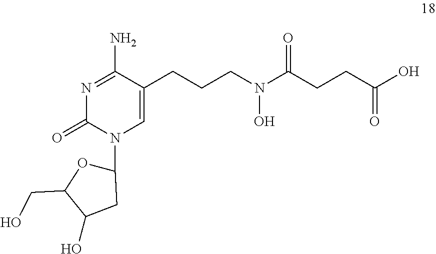 [SMILES: Nc1nc(=O)n(C2CC(O)C(CO)O2)cc1CCCN(O)C(=O)CCC(=O)O]

White solid (51% yield). ¹H NMR (METHANOL-d₄, 500 MHz): d=7.82 (s, 1H), 6.18 (t, J=6.6 Hz, 1H), 4.27-4.35 (m, 1H), 3.81 (q, J=3.7 Hz, 1H), 3.75 (dd, J=12.1, 3.2 Hz, 1H), 3.65 (dd, J=12.1, 3.8 Hz, 1H), 3.55 (t, J=6.3 Hz, 2H), 2.58-2.63 (m, 2H), 2.44-2.50 (m, 2H), 2.19-2.26 (m, 3H), 2.05-2.12 (m, 1H), 1.69-1.78 ppm (m, 2H). (ESI-TOF) m/z [M−H]⁻ calculated for C₁₆H₂₃N₄O₈⁻ 399.2, found 399.2.

### Example 2. Biological Assays

### Materials and Methods

All chemicals and reagents required for the synthesis, purification, and assaying of TET inhibitors were purchased from Sigma Aldrich unless otherwise stated. NMR and LCMS solvents were obtained from Fisher Scientific. K562, HEK293T, and A549 cells used for cell-based assays were obtained from ATCC or the Cleveland Clinic. All materials for tissue culture and related experiments were obtained from Corning and Gibco. DNA extraction kits were obtained from Qiagen. Recombinant TET1 (#31417) and TET2 (#31418) enzymes were purchased from Active Motif. Synthetic DNA oligomer substrates were ordered from Integrated DNA Technologies. Internal standards for LCMS quantitation of 5mC and 5hmC were obtained from Toronto Research Chemicals. Nucleotide digestion buffer was supplied by New England Biolabs (#M0649S). Filters used for purification of nucleotides were acquired from Pall Corporation. Licor Image Studio was used to generate process acquired dot blotting images and all statistical analyses herein were performed with GraphPad Prism v9.3.1.

### Cell Culture and Treatment

Cells were grown in MEM media supplemented with 10% FBS (Gibco by Life Technologies TM, #16140-071) and AntibioticAntimycotic (Gibco by Life Technologies TM, #15240062) at 37° C. under 5% CO₂. Solutions of inhibitors were prepared in dimethyl sulfoxide (DMSO) were incubated with cells at the specific concentration and duration prior to collection and extraction of genomic DNA.

### In-Vitro TET Assays

For in vitro TET assays, 10 pmol of 16-mer hemi-methylated synthetic dsDNA oligomer designed after the Rassfla tumor suppressor gene promotor (5′-AATTAGAA[5meC]GCTCCTT3′) was incubated with 2×TET reaction buffer (50 mM HEPES [pH 7.9], 100 mM NaCl, 75 μM Fe(NH₄)₂(SO₄)₂, 2 mM ascorbate, 10 μM α-ketoglutarate, 1 mM DTT), recombinant human TET1 or TET2 catalytic domain (0.2 μg, 2.3 pmol, 115 nM) and 5% DMSO (or inhibitor in DMSO) on a 20 μL scale. Timing was achieved by withholding protein until the ‘start’ of the incubation window. Reactions were completed on a dry block at 37° C. for 30 minutes. Reactions were terminated at the ‘end’ of the incubation window by transferring reaction tubes to dry ice to snap-freeze the reaction until all replicates were completed. Upon completion, all samples were heat-inactivated at 90° C. for 10 minutes and then dried entirely in SpeedVac concentrator. Sample were then prepared for LCMS or dot blot analysis. HPLC-ESI-MS/MS quantitation of 5mC and 5hmC.

Quantitation of 5hmC is based on methodology previously described in Seiler et al., with slight modification.²¹ Dried enzymatic reaction samples were reconstituted with mQ-water and spiked with 1 pmol each of 5-Methyl-2′-deoxycytidine-d₃ and 5-(Hydroxymethyl)-2′deoxycytidine-d₃. Nucleotide digestion enzyme mix and buffer were added per product guidelines on a 20 μL scale. Samples were digested for 60 min at 37° C. on a dry block. Following digestion, samples were filtered on nanosep 10K Omega filters. The samples were then dried entirely and resuspended in 100 μL mQ-water for offline HPLC cleanup to enrich 5hmC. An Atlantis T3 column (Waters, 4.6×150 mm, 3 μm) was eluted at a flow of 0.9 mL/min with a linear gradient previously described of 5 mM NH₄CHO₂ buffer, pH 4.0 (A) and MeOH (B). Fractions containing 5meC and 5hmC were collected and pooled before analysis by isotope dilution HPLC-ESI-MS/MS. Pooled samples were dried and reconstituted in 2 mM ammonium formate buffer (NH₄CHO₂) with a Dionex Ultimate 3000UHPLC (Thermo Fisher) interfaced with a Thermo TSQ Quantis mass spectrometer (Thermo Fisher) flowing 2 mM NH₄CHO₂ (A) and ACN (B) at 15 μL/min through a Zorbax SB-C18 column (Agilent, 0.5×150 mm, 3 μm) with the gradients as previously described. 5meC and 5-d₃-meC eluted at 5.1 minutes while 5hmC and 5-d₃-hmC eluted at 3.5 minutes. Quantitation was completed monitoring MS/MS transitions m/z 242.1 [M+H⁺]→m/z 126.1 [M-deoxyribose+H⁺] for 5meC, m/z 245.2 [M+H⁺]→m/z 129.0 [M-deoxyribose+H⁺] for 5-d₃-meC, m/z 258.1 [M+H⁺]→m/z 142.1 [M-deoxyribose+H⁺] for 5hmC, m/z 261.1 [M+H⁺]→m z 145.1 [M-deoxyribose+H⁺] for 5-d₃-hmC. All mass spectrometry parameters were optimized through direct infusion of authentic standards with settings as follows: spray voltage of 2700 V, sheath gas of 15 au, source fragmentation voltage of 5 V, and ion transfer tube temperature of 350° C. Fragmentation was induced with an Argon gas flow of 1 mTorr.

### DNA Dot Blotting

For cellular assays, genomic DNA was extracted using DNA Blood/Tissue Kit, denatured, and subjected to dot blotting analysis using antibody against 5hmC (Active Motif, #39769) as previously described.²² Briefly, 2 μg of purified DNA was denatured at 100° C. and mixed 1:1 with 2M NaOAc. DNA was loaded to the membrane, washed, and blocked with 5% NFM. Primary antibody for 5-hmC was incubated overnight and detected by fluorescence. The DNA spotted membrane was then stained with 0.02% methylene blue in 0.5 M sodium acetate (pH 5.0) for DNA loading control. The same protocol was followed for in vitro assays.

### Results

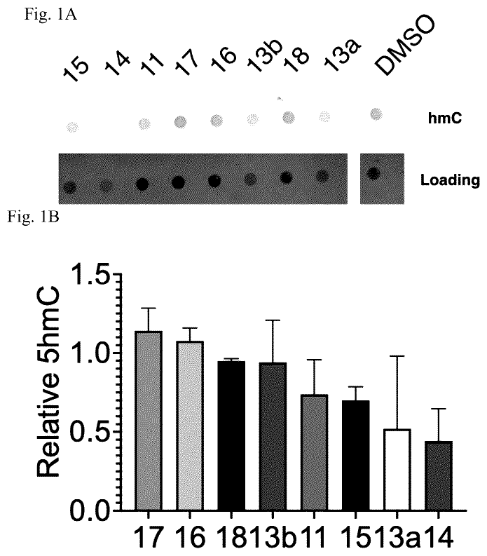

K562 cells, a TET proficient leukemia model, were treated with 5 μM of respective compound for 24 hours. Genomic DNA was extracted and 5hmC levels were quantified via dot blot (FIG. 1A and FIG. 1B). The most active compounds were, compounds 11, 13b 14, and 15, which lowered the relative levels of 5hmC by 30±8%, 55±20%, 26±21%, and 47±45%, respectively. These data revealed interesting SAR trends, namely that the substrate mimetic portion of the molecule prefer a 2′-deoxycytidine scaffold opposed to 2′-deoxyuridine and that a pentyne based linker was optimal, which correlated with docking studies.

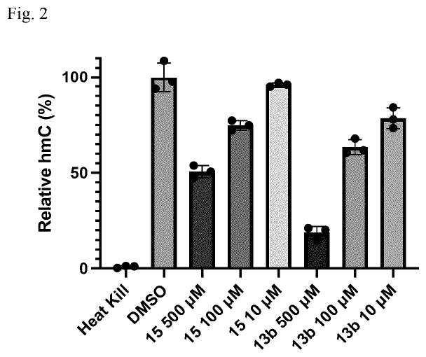

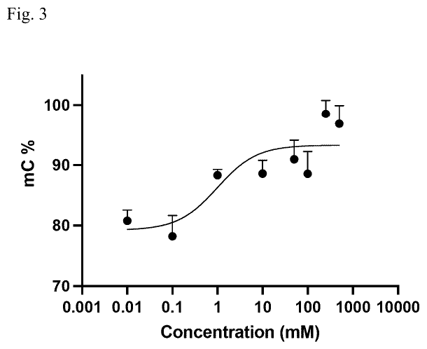

Compounds 13b and 15 were taken forward and incubated with recombinant TET1 and TET2 protein to further evaluate their potential TET inhibitory activity. The compounds 13b and 15 were taken forward and evaluated in an in vitro TET activity assay with the enzymatically active catalytic domain of human TET1 using a hemimethylated synthetic oligomer substrate. Following a 30 minute enzymatic incubation time in the presence or absence of inhibitor, the reactions were halted through denaturation, and levels of 5mC 5hmC were quantified via LCMS/MS via direct isotope dilution (Seiler, C. L., et al., Biochemistry 2018, 57 (42), 6061-6069). As shown in FIG. 2, both compounds 13b and 15 displayed dose-dependent TET inhibition. Of the two, the more potent compound 13b, reduced hmC production by 85±3%, 30±4%, 22±5%, at 500 μM, 100 μM, 10 μM, respectively, in comparison to DMSO control. These data indicate that this class of bifunctional substrate cofactor mimic compounds possess mid μM TET1 inhibitory activity.

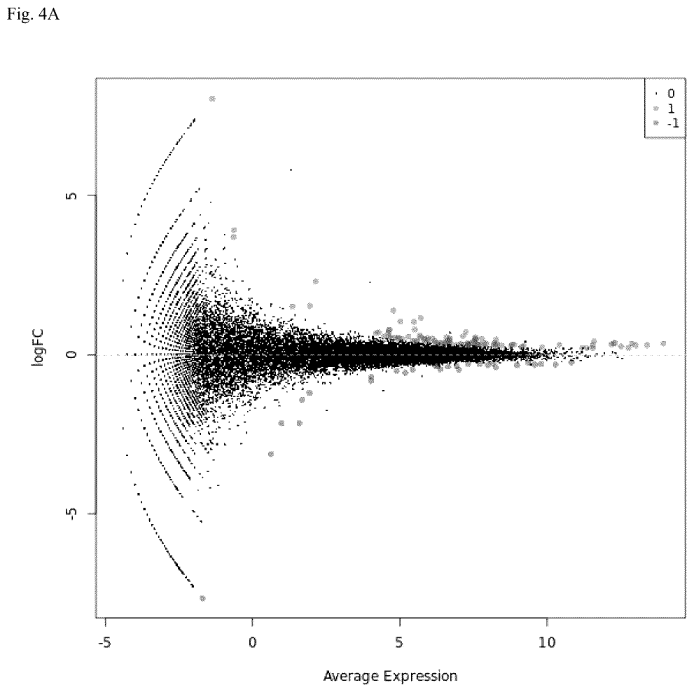

Compound 13b was also tested with TET2 is the same assay described above. As shown in FIG. 3, this compound inhibits TET2 mediated conversation of 5mC to 5hmC in a dose dependent manner with an IC50 value ˜1 mM.

### Example 3. Effect of TET Inhibitor 13b on the Patterns of Gene Expression as Determined by RNA-Seq

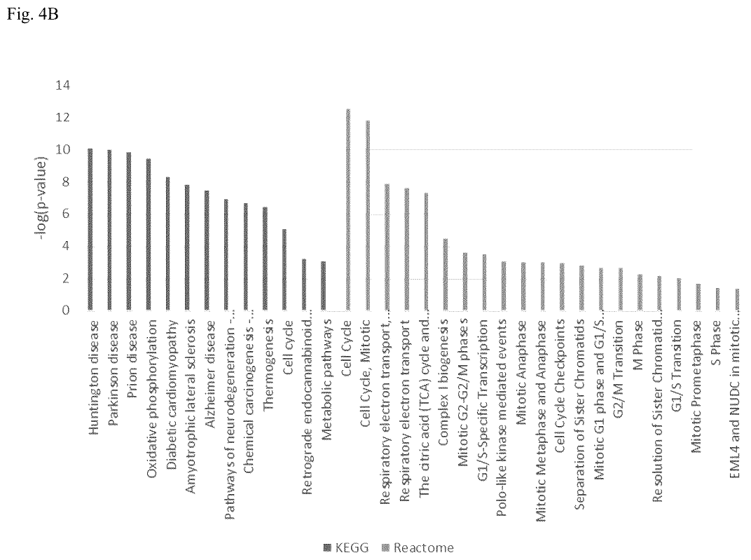

Lung adenocarcinoma cells (H_{441 1} million, in triplicate) were treated with 10 μM of comp 13a or DMSO control for 48 hours. RNA was extracted using the Qiagen AllPrep DNA/RNA Mini Kit (Qiagen, Hilden Germany) according to the manufacturer's instructions and quantified using Qubit fluorometric assay (Thermo Fisher Scientific, Fairlawn, NJ). RNA integrity was confirmed using Agilent Bioanalyzer (Agilent, Santa Clara CA). Total RNA samples were converted to Illumina sequencing libraries using SMARTer Stranded Total RNA-Seq Kit-Pico Mammalian Input (Takara Bio USA, Mountain View CA). RNA was reverse transcribed into cDNA, and Illumina adapters were added using PCR. Sequencing was performed on the Illumina HiSeq 2500 sequencing system using Illumina's SBS chemistry. Data analysis: Raw RNASeq FASTAQ files were QC′d via trimmomatic before being aligned to the human genome via HISAT2 and counts generated via feature Counts; these counts were then fed into edgeR for differential expression analysis. Data is shown in FIG. 4A and FIG. 4B. The resulting significantly upregulated and downregulated genes were then fed into gProfiler for Gene Ontology.

### Example 4

The following illustrate representative pharmaceutical dosage forms, containing a compound of formula (I) (‘Compound X’), for therapeutic or prophylactic use in humans.

| (i) Tablet 1 | mg/tablet |
|---|---|
| Compound X= | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |

| (ii) Tablet 2 | mg/tablet |
|---|---|
| Compound X= | 20.0 |
| Microcrystalline cellulose | 410.0 |
| Starch | 50.0 |
| Sodium starch glycolate | 15.0 |
| Magnesium stearate | 5.0 |
| | 500.0 |

| (iii) Capsule | mg/capsule |
|---|---|
| Compound X= | 10.0 |
| Colloidal silicon dioxide | 1.5 |
| Lactose | 465.5 |
| Pregelatinized starch | 120.0 |
| Magnesium stearate | 3.0 |
| | 600.0 |

| (iv) Injection 1 (1 mg/ml) | mg/ml |
|---|---|
| Compound X = (free acid form) | 1.0 |
| Dibasic sodium phosphate | 12.0 |
| Monobasic sodium phosphate | 0.7 |
| Sodium chloride | 4.5 |
| 1.0N Sodium hydroxide solution | q.s. |
| (pH adjustment to 7.0-7.5) | |
| Water for injection | q.s. ad 1 mL |

| (v) Injection 2 (10 mg/ml) | mg/ml |
|---|---|
| Compound X = (free acid form) | 10.0 |
| Monobasic sodium phosphate | 0.3 |
| Dibasic sodium phosphate | 1.1 |
| Polyethylene glycol 400 | 200.0 |
| 1.0N Sodium hydroxide solution | q.s. |
| (pH adjustment to 7.0-7.5) | |
| Water for injection | q.s. ad 1 mL |

| (vi) Aerosol | mg/can |
|---|---|
| Compound X= | 20.0 |
| Oleic acid | 10.0 |
| Trichloromonofluoromethane | 5,000.0 |
| Dichlorodifluoromethane | 10,000.0 |
| Dichlorotetrafluoroethane | 5,000.0 |

The above formulations may be obtained by conventional procedures well known in the pharmaceutical art.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A compound of formula (I): 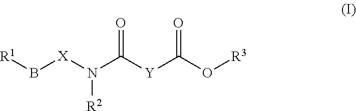
or a salt thereof, wherein:
R¹ is: 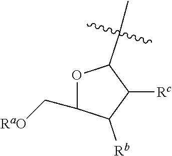
B is selected from the group consisting of: 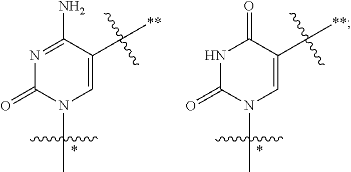
wherein * designates the point of attachment to R¹ and ** designates the point of attachment with X;
X is (C₁-C₆)alkyl or (C₂-C₆)alkynyl;
R² is H, OH, or (C₁-C₃)alkoxy;
Y is (C₁-C₆)alkyl or (C₂-C₆)alkenyl;
R³ is H or (C₁-C₆)alkyl;
R^{a} is H;
R^{b} is OH; and
R^{c} is H;
provided the compound of formula (I) is not:

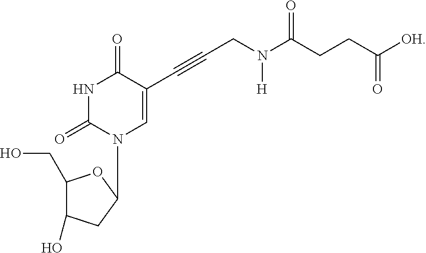 [SMILES: O=C(O)CCC(=O)NCC#Cc1cn(C2CC(O)C(CO)O2)c(=O)[nH]c1=O]

2. The compound or salt of claim 1, wherein the compound of formula (I) is not:

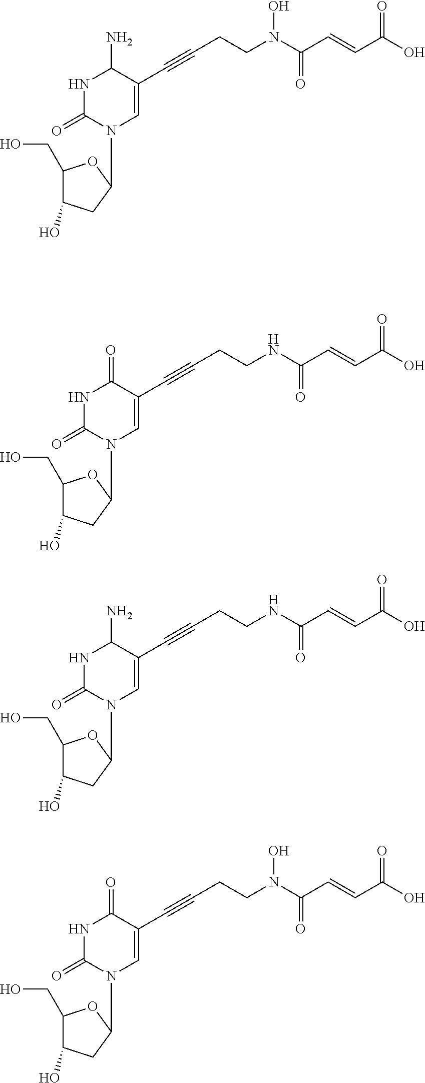 [SMILES: NC1NC(=O)N(C2C[C@H](O)C(CO)O2)C=C1C#CCCN(O)C(=O)/C=C/C(=O)O ; O=C(O)/C=C/C(=O)NCCC#Cc1cn(C2C[C@H](O)C(CO)O2)c(=O)[nH]c1=O ; NC1NC(=O)N(C2C[C@H](O)C(CO)O2)C=C1C#CCCNC(=O)/C=C/C(=O)O ; O=C(O)/C=C/C(=O)N(O)CCC#Cc1cn(C2C[C@H](O)C(CO)O2)c(=O)[nH]c1=O]

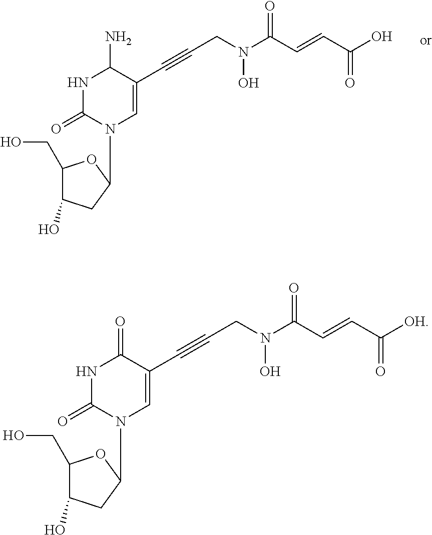 [SMILES: NC1NC(=O)N(C2C[C@H](O)C(CO)O2)C=C1C#CCN(O)C(=O)/C=C/C(=O)O ; O=C(O)/C=C/C(=O)N(O)CC#Cc1cn(C2C[C@H](O)C(CO)O2)c(=O)[nH]c1=O]

3. The compound or salt of claim 1, which is a compound of formula (Ia): 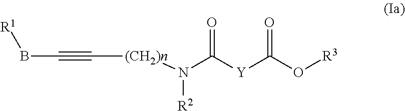
or a salt thereof, wherein n is 1, 2, 3, or 4.

4. The compound or salt of claim 1, wherein X is (C₁-C₆)alkyl.

5. The compound or salt of claim 1, wherein X is (C₂-C₆)alkynyl.

6. The compound or salt of claim 1, wherein R² is H.

7. The compound or salt of claim 1, wherein R² is OH.

8. The compound or salt of claim 1, wherein R² is (C₁-C₃)alkoxy.

9. The compound or salt of claim 1, wherein Y is (C₁-C₆)alkyl.

10. The compound or salt of claim 1, wherein Y is (C₂-C₆)alkenyl.

11. The compound or salt of claim 1, wherein R³ is H.

12. The compound or salt of claim 1, wherein R³ is (C₁-C₆)alkyl.

13. A pharmaceutical composition comprising a compound of formula (I): 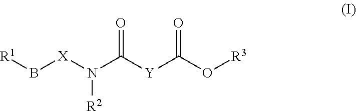
or a pharmaceutically acceptable salt thereof, wherein:
R¹ is: 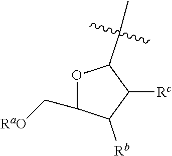
B is selected from the group consisting of: 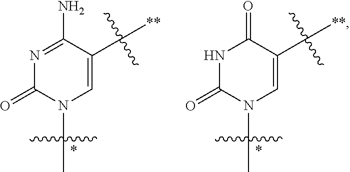
wherein * designates the point of attachment to R¹ and ** designates the point of attachment with X;
X is (C₁-C₆)alkyl or (C₂-C₆)alkynyl;
R² is H, OH, or (C₁-C₃)alkoxy;
Y is (C₁-C₆)alkyl or (C₂-C₆)alkenyl;
R³ is H or (C₁-C₆)alkyl;
R^{a} is H or,
R^{b} is OH or, and
R^{c} is H or,
and a pharmaceutically acceptable carrier.

14. The compound or salt of claim 1, which is selected from the group consisting of: 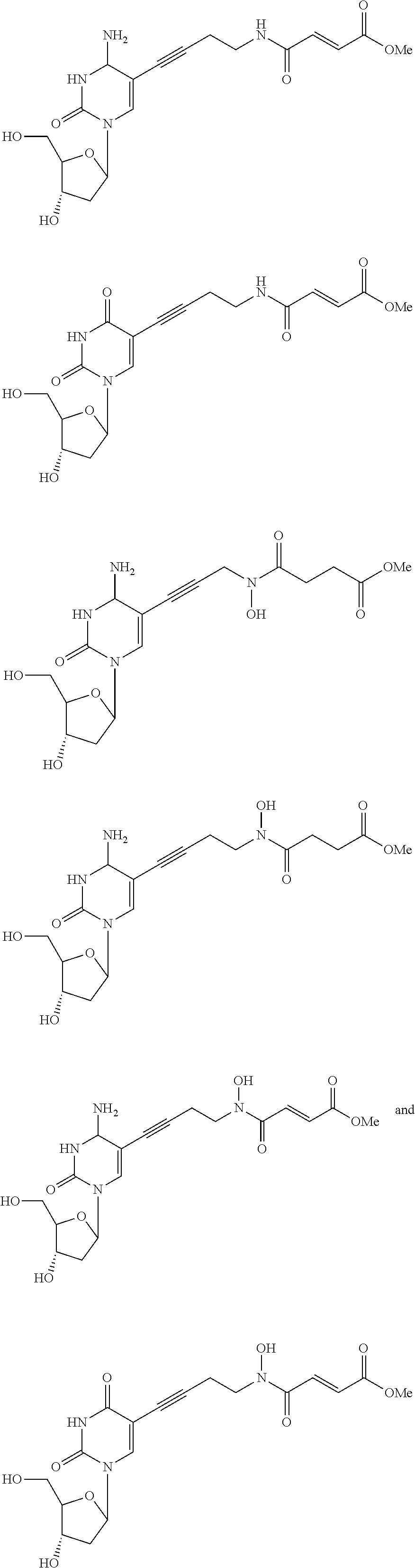 [SMILES: COC(=O)/C=C/C(=O)NCCC#CC1=CN(C2C[C@H](O)C(CO)O2)C(=O)NC1N ; COC(=O)/C=C/C(=O)NCCC#Cc1cn(C2C[C@H](O)C(CO)O2)c(=O)[nH]c1=O ; COC(=O)CCC(=O)N(O)CC#CC1=CN(C2C[C@H](O)C(CO)O2)C(=O)NC1N ; COC(=O)CCC(=O)N(O)CCC#CC1=CN(C2C[C@H](O)C(CO)O2)C(=O)NC1N ; COC(=O)/C=C/C(=O)N(O)CCC#CC1=CN(C2C[C@H](O)C(CO)O2)C(=O)NC1N ; COC(=O)/C=C/C(=O)N(O)CCC#Cc1cn(C2C[C@H](O)C(CO)O2)c(=O)[nH]c1=O]
and salts thereof.

15. The compound or salt of claim 1, which is selected from the group consisting of: 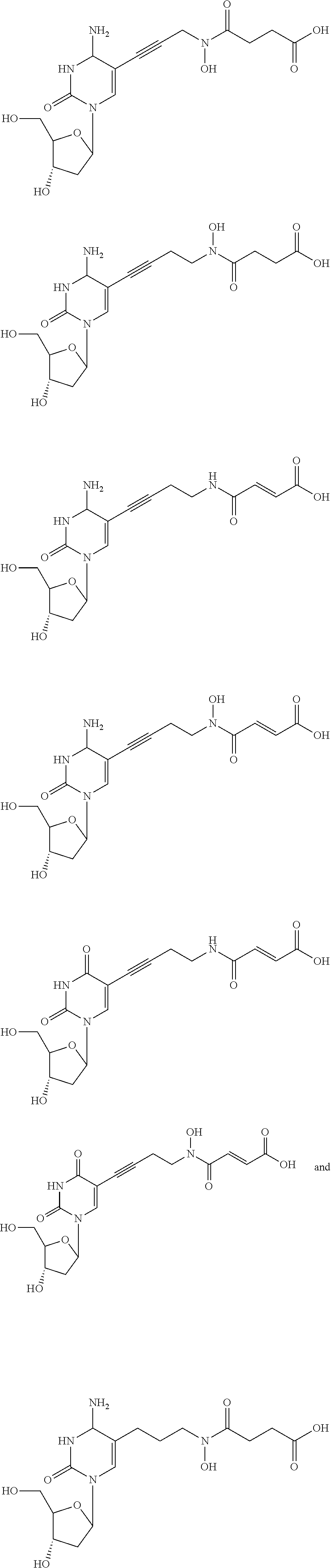
and salts thereof.

16. A pharmaceutical composition comprising a compound of formula (I): 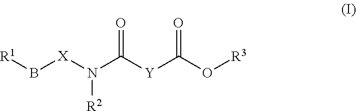
or a pharmaceutically acceptable salt thereof, salt thereof, wherein:
R¹ is: 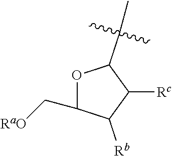
B is selected from the group consisting of: 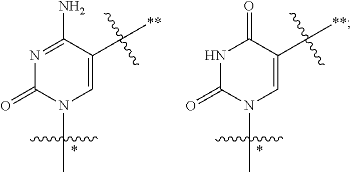
wherein * designates the point of attachment to R¹ and ** designates the point of attachment with X;
X is (C₁-C₆)alkyl or (C₂-C₆)alkynyl;
R² is H, OH, or (C₁-C₃)alkoxy;
Y is (C₁-C₆)alkyl or (C₂-C₆)alkenyl;
R³ is H or (C₁-C₆)alkyl;
R^{a} is H;
R^{b} is OH; and
R^{c} is H;
and a pharmaceutically acceptable carrier.

17. A method to treat cancer, a neurological disorder, an immunological disorder, or an eating disorder in an animal, comprising administering a compound of formula (I) as described in claim 1 or a pharmaceutically acceptable salt thereof to the animal.

18. A method to modulate DNA demethylation, comprising contacting a TET enzyme with a compound of formula (I) as described in claim 1 or a salt thereof in vitro or in vivo.

19. A method to inhibit TET activity in vitro or in vivo comprising contacting a TET enzyme with a compound of formula (I) as described in claim 1 or a salt thereof.

20. A method to improve the efficacy of immunotherapy for cancer in an animal, comprising administering a compound of formula (I) as described in claim 1 or a pharmaceutically acceptable salt thereof to patient cells in combination with the immunotherapy.
